(19) United States
(12) Patent Application Publication
(10) Pub. No.: US 2024/0425710 A1
(43) Pub. Date: Dec. 26, 2024

(54) PEPTIDE-BASED BIOINK COMPOSITIONS

(71) Applicant: Ramot at Tel-Aviv University Ltd., Tel-Aviv (IL)

(72) Inventors: Lihi ADLER-ABRAMOVICH, Tel-Aviv (IL); Francesca NETTI, Tel-Aviv (IL)

(73) Assignee: Ramot at Tel-Aviv University Ltd., Tel-Aviv (IL)

(21) Appl. No.: 18/822,661

(22) Filed: Sep. 3, 2024

Related U.S. Application Data
(63) Continuation of application No. PCT/IL2023/050219, filed on Mar. 3, 2023.
(60) Provisional application No. 63/316,060, filed on Mar. 3, 2022.

(51) Int. Cl. C09D 11/04; A61L 27/26; A61L 27/52; B29C 64/112; B29K 105/00; B29L 31/00; B33Y 10/00; B33Y 70/00; B33Y 80/00; C08L 89/06

(52) U.S. Cl. CPC C09D 11/04; A61L 27/26; A61L 27/52; B29C 64/112; B33Y 10/00; B33Y 70/00; B33Y 80/00; C08L 89/06; B29K 2089/00; B29K 2105/0061; B29K 2995/0056; B29L 2031/7532

(57) ABSTRACT

Bioink compositions comprising a plurality of aromatic peptides and a matrix-forming material, formulations and kits for preparing same, additive manufacturing (bioprinting) processes utilizing same and three-dimensional objects obtained thereby are provided.

### RELATED APPLICATIONS

This application is a Continuation of PCT Patent Application No. PCT/IL2023/050219 having International filing date of Mar. 3, 2023, which claims the benefit of priority under 35 USC § 119(e) of U.S. Provisional Patent Application No. 63/316,060, filed on Mar. 3, 2022. The contents of the above applications are all incorporated by reference as if fully set forth herein in their entirety.

### FIELD AND BACKGROUND OF THE INVENTION

The present invention, in some embodiments thereof, relates to material science, and more particularly, but not exclusively, to bioink compositions and bioprinting of three-dimensional objects using same.

One of the aims of regenerative medicine is to promote tissue regeneration by using biomaterials as a temporary scaffold that is then resorbed or degraded as the new tissue grows [D. F. Williams, Biomaterials, 2009, 30, 5897-5909; Mano et al., J. R. Soc. Interface, 2007, 4, 999-1030]. Whether composed of synthetic or natural materials, the ideal biomedical scaffold for this purpose should be designed to mimic the 3D extracellular matrix (ECM) structure and be able to maintain its structure and activity, while enhancing tissue regeneration [Place et al, Nat. Mater., 2009, 8, 457-470; Primo and Mata, Adv. Funct. Mater., 2021, 2009574].

Hydrogels can absorb several times their dry weight in water. This high water content, trapped in the hydrogel matrix, makes them suitable for several biomedical applications, such as drug delivery, cell adhesion, and proliferation [Molina et al, Biomaterials, 2001, 22, 363-369]. Hydrogels are often used as ECM-mimicking scaffolds for tissue engineering, utilizing natural components, which are highly biocompatible [Moroni et al, Nat. Rev. Mater., 2018, 3, 21-37; Schwab et al, Chem. Rev., 2020, 120, 11028-11055; Tytgat et al, Biomacromolecules, 2020, 21, 3997-4007; Firkowska-Boden, J. Mech. Behav. Biomed. Mater., 2021, 115, 1-8; Aviv et al., ACS Appl. Mater. Interfaces, 2018, 10, 41883-41891]. However, such biocompatible hydrogels are typically characterized by poor mechanical properties and therefore high concentrations are required [Schwab et al, 2020, supra].

Additive manufacturing (AM) is generally a process in which a three-dimensional (3D) object is manufactured utilizing a computer model of the objects. The basic operation of any AM system consists of slicing a three-dimensional computer model into thin cross sections, translating the result into two-dimensional position data and feeding the data to control equipment which manufacture a three-dimensional structure in a layerwise manner.

Various AM technologies exist, amongst which are stereolithography, digital light processing (DLP), and three-dimensional (3D) printing such as 3D inkjet printing. Such techniques are generally performed by layer by layer deposition and hardening of one or more building materials, which typically include photopolymerizable (photocurable) materials.

In three-dimensional printing processes, for example, a building material is dispensed from a dispensing head having a set of nozzles to deposit layers on a supporting structure. Depending on the building material, the layers may then be cured or solidified using a suitable device.

The building material preferably liquid at the working temperature at which it is dispensed, and subsequently hardened, typically upon exposure to curing energy (e.g., UV curing), to form the required layer shape.

Additive manufacturing has been first used in biological applications for forming three-dimensional sacrificial resin molds in which 3D scaffolds from biological materials were created.

3D bioprinting is an additive manufacturing methodology which uses biological or biocompatible materials, optionally in combination with chemicals and/or cells, that are printed layer-by-layer with a precise positioning and a tight control of functional components placement to create a 3D structure.

Recently, 3D bioprinting has attracted much attention due to the ability to design personalized scaffolds with unprecedented precision. Many efforts have been invested in fabricating new bioinks (bioink compositions) and developing new printing techniques suitable for tissue engineering [Yin et al., ACS Appl. Mater. Interfaces, 2018, 10, 6849-6857; You et al., Int. J. Polym. Mater. Polym. Biomater., 2017, 66, 299-306; Wang et al., Materials (Basel)., 2018, 11, 6-8; Chimene et al., ACS Appl. Mater. Interfaces, 2020, 12, 15976-15988]. 3D bioprinting allows the construction of a multi-layered scaffold with an architecture designed to provide the mechanical strength necessary for the mechano-sensing that stimulates cellular differentiation and regeneration [Ghiasi et al., Bone Reports, 2017, 6, 87-100; Pourchet et al., Adv. Healthc. Mater., 2017, 6, 1-8].

Three dimensional (3D) bioprinting is gaining momentum in many medicinal applications, especially in regenerative medicine, to address the need for complex scaffolds, tissues and organs suitable for transplantation.

Inherent to 3D printing in general is that the mechanical properties of the printing media (the dispensed building material) are very different from the post-printed cured (hardened) material.

Different technologies have been developed for 3D bioprinting, including 3D Inkjet printing, Extrusion printing, Laser-assisted printing and Projection stereolithography [see, for example, Murphy SV and Atala A, Nature Biotechnology, 2014, 32(8); Miller JS and Burdick J. ACS Biomater. Sci. Eng. 2016, 2, 1658-1661]. Each technology has its different requirements for the dispensed building material (e.g., bioink composition(s)), which is derived from the specific application mechanism and the hardening (e.g., curing) process required to maintain the 3D structure of the scaffold post-printing.

To allow tight control on the hardening after printing, the building material commonly includes polymerizable (e.g., photopolymerizable) moieties or groups that polymerize (e.g., by chain elongation and/or cross-linking) upon being dispensed, to preserve the geometric shape and provide the necessary physical properties of the final product.

For all AM technologies, the most important parameter determining the accuracy and efficiency of the printing is the static and dynamic physical properties of the dispensed building material, including viscosity, shear thinning and thixotropic properties. These properties facilitate printability and post-printing stability. The static and dynamic properties of the building material are important not only for the printing technology but also when considering cell-laden printing, i.e. including cells in the building material dispensed during printing. In this case, the shearing forces applied to the building material during printing (dispensing) have a significant effect on the survival of the cells. Therefore, it is desirable to have good control on the specific properties of the printing media over a wide range of conditions, such as concentration, temperature, ionic strength and pH.

Currently, different bioinks (bioink compositions) are commercially available for 3D-printing purposes. The printing process of most of these inks requires several potentially harmful agents for effecting, for example, UV-crosslinking or chemical polymerization, which compromise cell viability [Levato et al., Adv. Mater., 2020, 32, 1906423].

Collagen is the major component of the ECM and the different types of collagens present in the human body define the mechanical properties of specific organs [Boraschi-Diaz et al., Front. Phys., 2017, 5, 12]. Gelatin, a collagen derivative, is widely used as an adjuvant for 3D-printing applications in order to improve the viscosity and elasticity of materials and provide an unbroken matrix [Feng et al., Crit. Rev. Food Sci. Nutr., 2019, 59, 3074-3081; Godoi et al., J. Food Eng., 2016, 179, 44-54; Mu et al., Prog. Polym. Sci., 2021, 115, 101375]. The protein sequence includes the arginine-glycine-aspartic acid (RGD) motif, which plays a major role in cell adhesion [Yue et al., Biomaterials, 2015, 73, 254-271]. However, due to its inferior mechanical integrity and instability under physiological conditions, gelatin is usually strengthened and stabilized by the addition of hydrogels or by chemical modifications. Currently, the most common gelatin-based bioink is gelatin methacrylate (GelMA), which can be covalently UV cross-linked in order to stabilize the formed gelatin-based hydrogel at body temperature [Yue et al., 2015, supra; Ying et al., Bio-Design Manuf., 2018, 1, 215-224].

Additional background art includes Netti et al., Nanoscale, 2022, 14, 8525-8533; Raphael et al., Mater. Lett., 2017, 190, 103-106; Rauf et al., J. Mater. Chem. B, 2021, 9, 1069-1081; Ryan et al., Chem. Commun., 2011, 47, 475-477; Diaferia et al., Chem.—A Eur. J., 2018, 24, 6804-6817; Diaferia et al., Soft Matter, 2019, 15, 487-496; and Dias and Peng, Physiol. Behav., 2017, 176, 139-148.

### SUMMARY OF THE INVENTION

According to an aspect of some embodiments of the present invention there is provided a bioink composition comprising a matrix-forming biocompatible material and a self-assembled hydrogel formed of a plurality of peptides, wherein at least a portion of the plurality of peptides comprises aromatic peptides of 2 to 6 amino acid residues featuring an aromatic end-capping moiety.

According to some of any of the embodiments described herein, the plurality of peptides comprises at least one first portion that comprises the aromatic peptides of 2 to 6 amino acid residues featuring an aromatic end-capping moiety and at least a second portion that comprises aromatic peptides of 2 to 6 amino acid residues featuring an aromatic end-capping moiety and having an alkylene glycol-containing moiety conjugated thereto.

According to some of any of the embodiments described herein, at least a portion, or all, of the aromatic peptides in the first and/or second portion, if present, are aromatic dipeptides.

According to some of any of the embodiments described herein, at least a portion, or all, of the aromatic peptides in the first and/or second portion, if present, are aromatic homodipeptides.

According to some of any of the embodiments described herein, the aromatic homodipeptide is Phe-Phe.

According to some of any of the embodiments described herein, the aromatic homodipeptide is Fmoc-Phe-Phe.

According to some of any of the embodiments described herein, the alkylene glycol-containing moiety is an oligo(alkylene glycol) moiety of from 2 to 20, or from 2 to 10, or from 2 to 8, or from 2 to 6, alkylene glycol moieties.

According to some of any of the embodiments described herein, in at least a portion, or all, of the portion, the alkylene glycol-containing moiety is an oligo(ethylene glycol) moiety of from 2 to 20, or from 2 to 10, or from 2 to 8, or from 2 to 6, ethylene glycol moieties.

According to some of any of the embodiments described herein, a weight ratio of the first and second portions ranges from 5:1 to 1:5, or from 3:1 to 1:3, or from 3:1 to 1:1.

According to some of any of the embodiments described herein, the matrix-forming biocompatible material is selected from a synthetic polymer, a naturally-occurring polymer, a protein and a carbohydrate.

According to some of any of the embodiments described herein, the matrix-forming biocompatible material is or comprises gelatin.

According to some of any of the embodiments described herein, a weight ratio of the matrix-forming agent and the self-assembled hydrogel ranges from 1:1 to 100:1.

According to some of any of the embodiments described herein, the bioink further comprises a biological component other than the matrix-forming biocompatible material and/or the aromatic peptides.

According to an aspect of some embodiments of the present invention there is provided a method of additive manufacturing a three-dimensional biocompatible object, the method comprising dispensing at least one bioink composition to sequentially form a plurality of layers in a configured pattern corresponding to a shape of the object, wherein for at least a portion of the layers, the dispensing is of the bioink composition as described herein in any of the respective embodiments and any combination thereof, thereby manufacturing the three-dimensional object.

According to an aspect of some embodiments of the present invention there is provided a three-dimensional object prepared by the method as described herein in any of the respective embodiments and any combination thereof.

According to an aspect of some embodiments of the present invention there is provided a three-dimensional object comprising a composition which comprises a matrix-forming biocompatible material and a self-assembled hydrogel formed of a plurality of peptides, the plurality of peptides comprising at least a first portion that comprises aromatic peptides of 2 to 6 amino acid residues featuring an aromatic end-capping moiety and at least a second portion that comprises aromatic peptides of 2 to 6 amino acid residues featuring an aromatic end-capping moiety and having an alkylene glycol-containing moiety conjugated thereto, the composition being shaped in a configured pattern corresponding to the shape of the three-dimensional object. The object according to these embodiments can be a printed object, prepared by an additive manufacturing (e.g., bioprinting) as described herein, or can be otherwise prepared from a bioink composition as described herein.

According to some of any of the embodiments described herein, the three-dimensional object further comprises a biological component associated with the composition.

According to some of any of the embodiments described herein, the biological component comprises cells.

According to an aspect of some embodiments of the present invention there is provided a formulation comprising a plurality of peptides which comprises at least a first portion comprising aromatic peptides of 2 to 6 amino acid residues featuring an aromatic end-capping moiety and at least a second portion comprising aromatic peptides of 2 to 6 amino acid residues featuring an aromatic end-capping moiety and having an alkylene glycol-containing moiety conjugated thereto, the plurality of peptides are capable of self-assembling to thereby form a hydrogel.

According to some of any of the embodiments described herein, a weight ratio of the first and second portions ranges from 5:1 to 1:5, or from 3:1 to 1:3, or from 3:1 to 1:1.

According to some of any of the embodiments described herein, preparing the ink composition comprises contacting the formulation with an aqueous solution, to thereby form the hydrogel.

According to some of any of the embodiments described herein, formulation is for use in preparing an ink composition that further comprises a matrix-forming material.

According to some of any of the embodiments described herein, the matrix-forming material is a biocompatible matrix-forming material, the formulation being for use in preparing a bioink composition.

According to some of any of the embodiments described herein, preparing the ink composition comprises contacting the formulation with an aqueous solution that comprises the matrix-forming agent.

According to an aspect of some embodiments of the present invention there is provided a method of preparing an ink composition that comprises a biocompatible matrix-forming material, the method comprising contacting the formulation of claim 19 or 20 and the biocompatible matrix-forming material with an aqueous solution.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of the molecular structures of exemplary aromatic peptides according to some of the embodiments of the present invention; Fmoc-FF, Fmoc-FF-EG₂-COOH (PepA) and Fmoc-FF-EG₆-COOH (PepB).

FIGS. 2A-F present photographs of inverted tubes of exemplary pristine and co-assembled hydrogels formed of formulations containing the exemplary aromatic peptides Fmoc-FF, PepA and PepB (FIG. 2A), and of hybrid hydrogels formed of formulations containing Fmoc-FF:PepA (FIG. 2B) and Fmoc-FF:PepB (FIG. 2C) at different ratios after overnight gelation, and transmission electron microscope (TEM) images of the formed hydrogels (FIG. 2D), and of the formed hybrid hydrogels of Fmoc-FF:PepA (FIG. 2E) and Fmoc-FF:PepB (FIG. 2F) at different ratios. Scale bar is 200 nm.

FIGS. 3A-B present data obtained in time sweep measurements, showing the measured (storage modulus) and G″ (loss modulus) of exemplary hydrogels according to some embodiments (5 g-L-1) formed of Fmoc-FF, Fmoc-FF:PepA, and of hybrid hydrogels thereof at different ratios (FIG. 3A) and of hydrogels formed of Fmoc-FF, Fmoc-FF:PepB, and of hybrid hydrogels thereof at different ratios (FIG. 3B).

FIGS. 4A-E present data obtained in thixotropic measurements as described herein, showing the G′ (storage modulus) and G″ (loss modulus) of the exemplary hydrogels according to some embodiments of the present invention, formed of Fmoc-FF (FIG. 4A), Fmoc-FF:PepA (1:1) (FIG. 4B), 1:1 Fmoc-FF:PepB (FIG. 4C), Fmoc-FF:PepA (3:1) (FIG. 4D) and 3:1 Fmoc-FF:PepB (FIG. 4E) at 5 g·L⁻¹, after 6 hour gelation.

FIGS. 5A-B present data obtained in rheological analyses of an exemplary Fmoc-FF hydrogel, measured in dynamic frequency sweep oscillatory test performed at 0.5% strain (FIG. 5A) and dynamic strain sweep performed at 5 Hz frequency (FIG. 5B).

FIGS. 6A-D present data obtained in rheological analyses as described herein, of exemplary Fmoc-FF:PepA hybrid hydrogels at ratios of 1:1 and 3:1, showing the G′ and G″ measured in dynamic frequency sweep oscillatory test performed at 5 Hz frequency (FIGS. 6A-B, respectively), and the G′ and G″ measured in dynamic strain sweep oscillatory test performed at 0.5% strain (FIGS. 6C and 6D, respectively).

FIGS. 7A-D present data obtained in rheological analyses as described herein, of exemplary Fmoc-FF:PepB hybrid hydrogels at ratios of 1:1 and 3:1, showing the G′ and G″ measured in dynamic frequency sweep oscillatory test performed at 5 Hz frequency (FIGS. 7A-B, respectively), and the G′ and G″ measured in dynamic strain sweep oscillatory test performed at 0.5% strain (FIGS. 7C and 7D, respectively).

FIGS. 8A-B present data obtained in measurements of assembly kinetics by hydrogels absorbance characterization, showing turbidity changes (absorbance at 400 nm) over time of hydrogels made of Fmoc-FF, PepA, compared to hybrid hydrogels formed of Fmoc-FF and PepA at various ratios (FIG. 8A) and of hydrogels made of Fmoc-FF, PepB, compared to hybrid hydrogels formed of Fmoc-FF and PepB at various ratios (FIG. 8B).

FIGS. 9A-C are photographs obtained during 3D-bioprinting of 5 grams·L⁻¹ exemplary hydrogels (bioinks) made of Fmoc-FF (FIG. 9A), 1:1 Fmoc-FF:PepA (FIG. 9B) and 1:1 Fmoc-FF:PepB (FIG. 9C).

FIG. 10 is a schematic illustration of the 3D-bioprinting process of exemplary composite hybrid hydrogels.

FIGS. 11A-B are photographs obtained in an injectability test, in which Fmoc-FF hydrogel (FIG. 11A) and Fmoc-FF/Gel composite hydrogel (FIG. 11B), prepared with the addition of blue food colorant, were loaded into a 1 mL syringe and injected in a 20 mL glass vial containing DDW.

FIGS. 12A-C are photographs obtained during 3D-printing of the exemplary gelatin (Gel)-containing composite hydrogel Fmoc-FF/Gel (FIG. 12A) and the exemplary Gel-based composite hybrid hydrogels Fmoc-FF:PepA (1:1)/Gel (FIG. 12B) and Fmoc-FF:PepB (1:1)/Gel (FIG. 12C).

FIGS. 13A-C present data obtained in time sweep analyses of gelatin (abbreviated as “Gel”) and the exemplary gelatin-containing composite hydrogel Fmoc-FF/Gel (FIG. 13A), and of the exemplary composite hybrid hydrogels Fmoc-FF:PepA/Gel (FIG. 13B) and Fmoc-FF:PepB/Gel (FIG. 13C) at different Fmoc-FF:PepA or Fmoc-FF:PepB ratios.

FIGS. 14A-G are SEM images of the exemplary hydrogel Fmoc-FF (FIG. 14A; Scale bar is m), the exemplary composite hydrogels Fmoc-FF/Gel (FIG. 14B), Gelatin (FIG. 14C), and the exemplary composite hybrid hydrogels 1:1 Fmoc-FF:PepA/Gel (FIG. 14D), 3:1 Fmoc-FF:PepA/Gel (FIG. 14E) and 1:1 Fmoc-FF:PepB/Gel (FIG. 14F), and 3:1 Fmoc-FF:PepB/Gel (FIG. 14G) hydrogels. Scale bar is 50 m unless indicated otherwise.

FIGS. 15A-G present data obtained in temperature-dependence analyses before (FIGS. 15A-C) and after (FIGS. 15D-F) 3D-printing of Gelatin (Gel) (FIG. 15A); an exemplary composite hydrogel Fmoc-FF/Gel (FIGS. 15A and 15D); an exemplary composite hybrid hydrogel Fmoc-FF:PepA/Gel (FIGS. 15B and 15E); and an exemplary composite hybrid hydrogel Fmoc-FF:PepB/Gel (FIGS. 15C and 15F), and a bar graph showing the storage modulus values (G′) of the exemplary composite hydrogels and composite hybrid hydrogels as measured at 25° C. and 37° C. (FIG. 15G).

FIG. 16 presents photographs of inverted tubes of a hydrogel made of gelatin (Gel), of a composite hydrogel Fmoc-FF/Gel; and of hybrid composite hydrogels Fmoc-FF:PepA (1:1 or 3:1)/Gel and Fmoc-FF:PepB (1:1 or 3:1)/Gel, as indicated), after overnight gelation at 25° C., before (25° C.) and after (37° C.) incubation at 37° C. for 20 minutes.

FIGS. 17A-D present optical images (FIGS. 17A-B) of 3D-printed objects made of gelatin (Gel) hydrogel and of an exemplary composite hydrogel Fmoc-FF/Gel (FIG. 17A), and of exemplary composite hybrid hydrogels Fmoc-FF:PepA (1:1 or 3:1)/Gel and Fmoc-FF:PepB (1:1 or 3:1)/Gel (FIG. 17B), as indicated, using a 22G nozzle and a 27G nozzle; Scale bar 2 mm, and bar graphs (FIGS. 17C-D) showing filament spreading ratio analyses of the 3D-printed objects upon extrusion through 22G (FIG. 17C) and 27G (FIG. 17D) nozzles; data analyzed using One-way ANOVA test (* p<0.05 and **** p<0.0001).

FIGS. 18A-B are bar graphs showing cell viability (%) following staining of 3T3 mouse fibroblasts (FIG. 18A) and MP902 murine stem cell (FIG. 18B) with alamarBlue™, 24 hours (24h) and 48 hours (48h) after seeding on 3D-printed objects made of the exemplary composite hybrid hydrogels Fmoc-FF:PepA (3:1)/Gel and Fmoc-FF:PepB (3:1)/Gel. Data were analyzed using One-way ANOVA test. * p<0.05 and **** p<0.0001.

FIG. 19 presents photographs showing the degradability of 3D-printed objects made of exemplary composite hybrid hydrogels Fmoc-FF:PepA (3:1 or 1:1)/Gel and Fmoc-FF:PepB (3:1 or 1:1)/Gel, as indicated. Images were obtained before (denoted “Room Temperature”) and after incubation at 37° C. in PBS for a duration of 24 hours (24h) and 48 hours (48h). The striped markings highlight the 3D-printed region and its remaining after degradation.

FIGS. 20A-B present confocal microscopy images of LIVE/DEAD™ assay of 3T3 mouse fibroblasts (FIG. 20A) and MP902 murine stem cell (FIG. 20B), as described, 48 hours after seeding on 3D-printed objects made of the exemplary composite hybrid hydrogels Fmoc-FF:PepA (3:1)/Gel, Fmoc-FF:PepB (3:1)/Gel, as indicated. Scale bar is 200 m.

### DESCRIPTION OF SPECIFIC EMBODIMENTS OF THE INVENTION

The present invention, in some embodiments thereof, relates to material science, and more particularly, but not exclusively, to bioink compositions and bioprinting of three-dimensional objects using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Over the last decade, three-dimensional (3D) printing technologies have attracted the interest of researchers due to the possibility of fabricating tissue- and organ-like contracts with structural similarities to the original organ. One of the most widely used materials for the fabrication of bioinks is gelatin (Gel) due to its excellent biocompatibility properties.

In order to fabricate stable scaffolds under physiological condition, the current most common approach employs gelatin methacrylate (GelMA), which undergoes UV crosslinking to stabilize it. However, the crosslinking process can be harmful to cell viability.

In a search for 3D-bioprinting methodology that can circumvents the need for post-printing crosslinking, the present inventors have conceived using self-assembling peptide-based formulations that provide bioink compositions made of self-assembled hydrogels. While the peptide-based bioink compositions were found to lack the mechanical and rheological properties that are required for 3D-bioprinting methodologies, combining such formulations with a matrix-forming material such as gelatin provided composite hydrogel-containing bioink compositions that meet the 3D-bioprinting process requirements and provide 3D-printed objects that feature the desired biocompatibility and mechanical properties.

While reducing the present invention to practice, the present inventors have designed self-assembling peptide-based formulations for use in the preparation for bioink compositions that feature mechanical and rheological properties that meet the 3D-bioprinting process requirements.

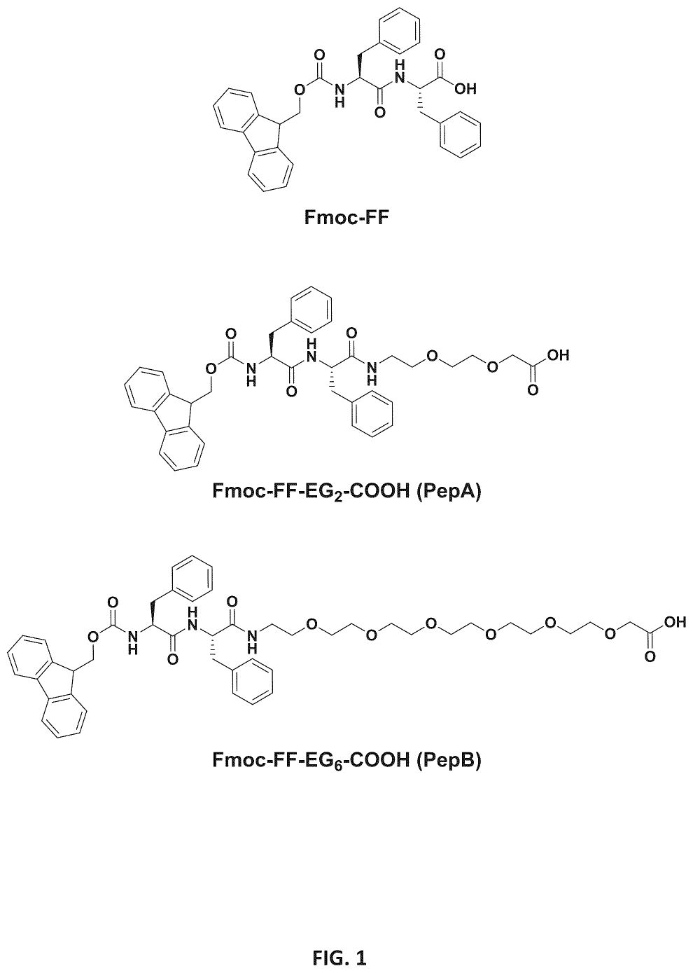

The present inventors have utilized, as an exemplary formulation, an aqueous solution of the homodipeptide Fmoc-FF, optionally in combination with a conjugate of the homodipeptide with an alkylene glycol moiety, such as shown in FIG. 1. The present inventors have studied the effect of various conjugates and various combinations of the homopeptide and the various conjugates on the mechanical and rheological properties of bioink compositions formed of such formulations.

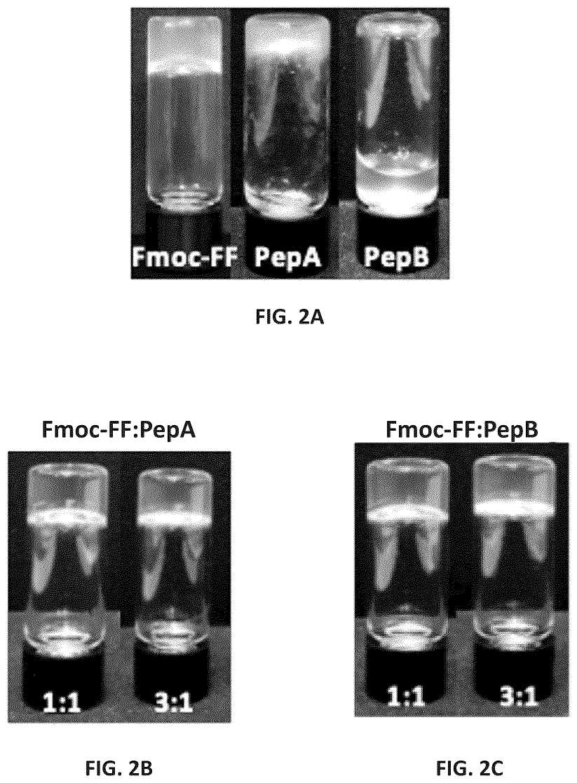

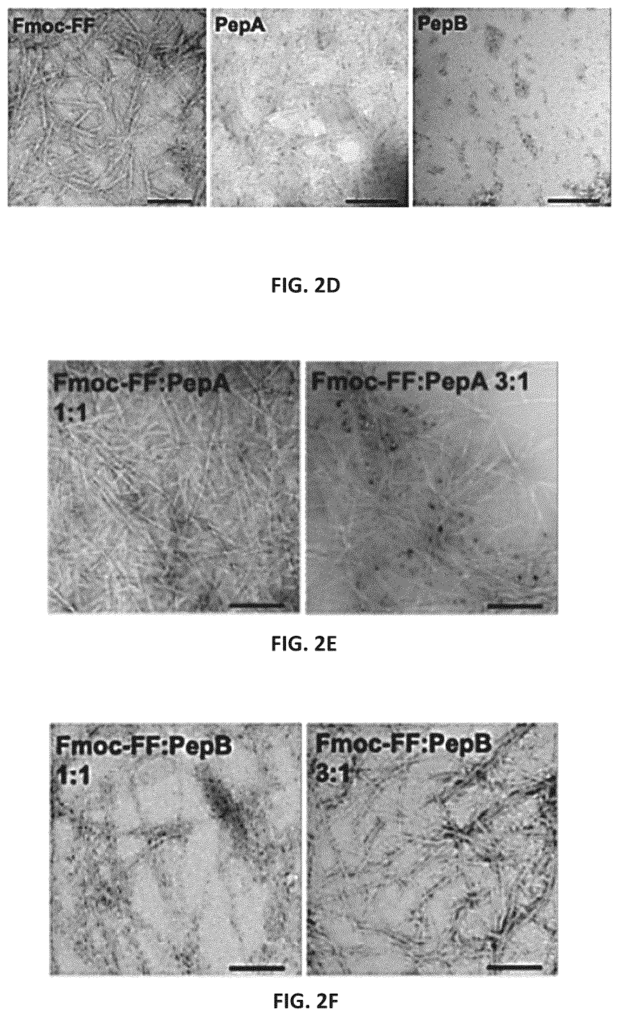

FIGS. 2A-F, 3A-B, 4A-E, 5A-B, 6A-D, 7A-D and 8A-B present the data obtained in these studies.

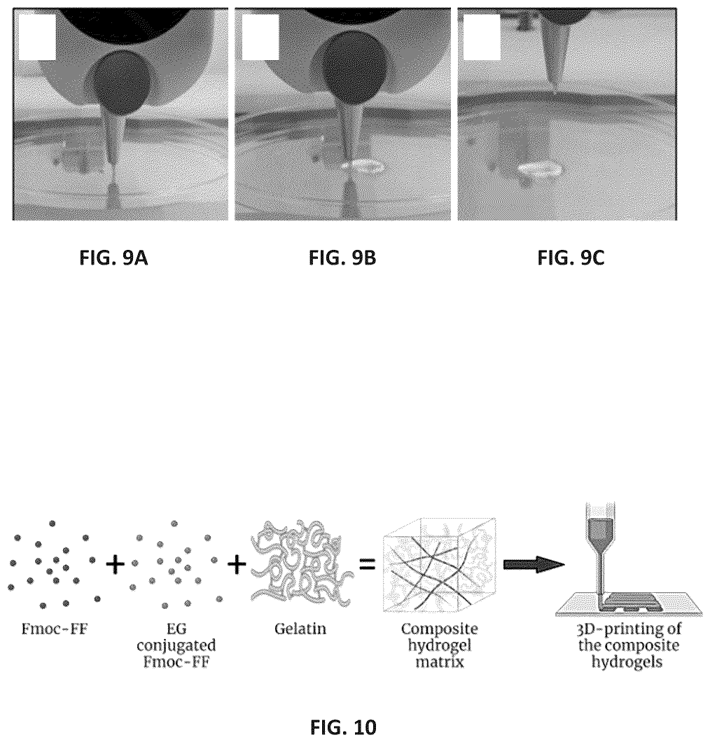

The present inventors have uncovered that while the length of the conjugated alkylene glycol moiety and the relative portion of the conjugate in the formulation affect the mechanical and rheological properties of bioink compositions comprising same, the obtained compostions do not meet the requirements of bioprinting processes (see, FIGS. 9A-C), and have conceived combining the practiced formulations with a matrix-forming biocompatible material that is commonly used in bioprinting and in therapeutical applications in general; gelatin, as schematically illustrated in FIG. 10. The present inventors have studied the effect of such a combination also in terms of enabling the use of biocompatible materials such as gelatin in 3D bioprinting, while circumventing the need for post-printing procedures, such as application of a curing energy (e.g., irradiation). As discussed herein, currently practiced 3D bioprinting methodologies that involve matrix-forming biocompatible materials such as gelatin, typically utilized curable derivatives of such materials, such as, for example, GelMa (gelatin methacrylate), which require post-printing irradiation of the printed object in order to achieve a self-supporting matrix.

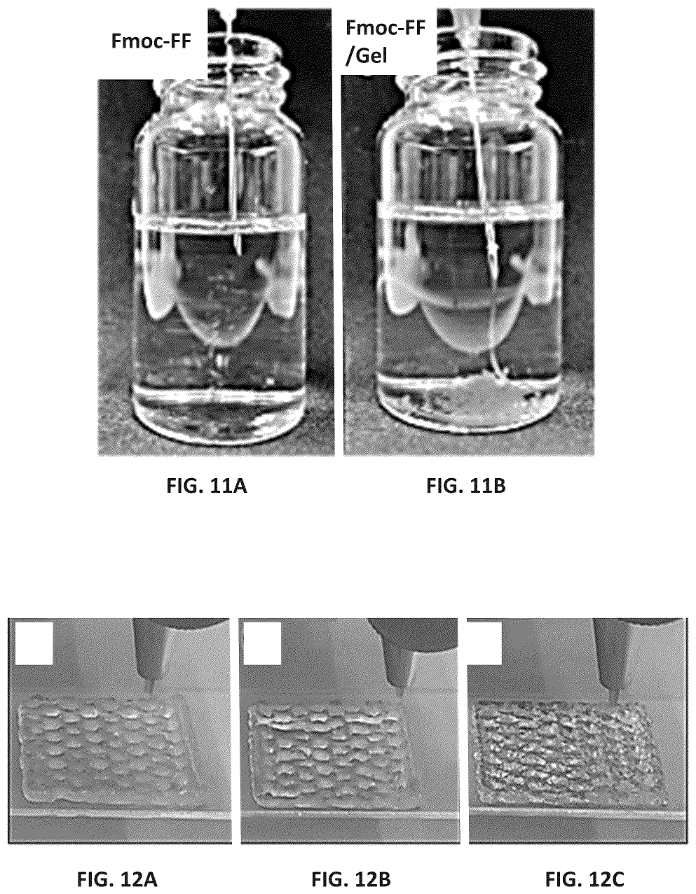

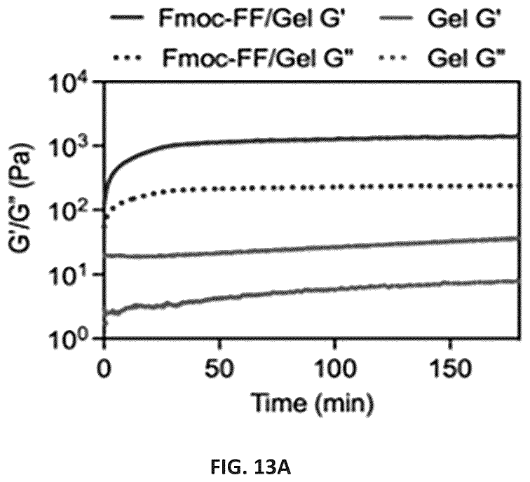

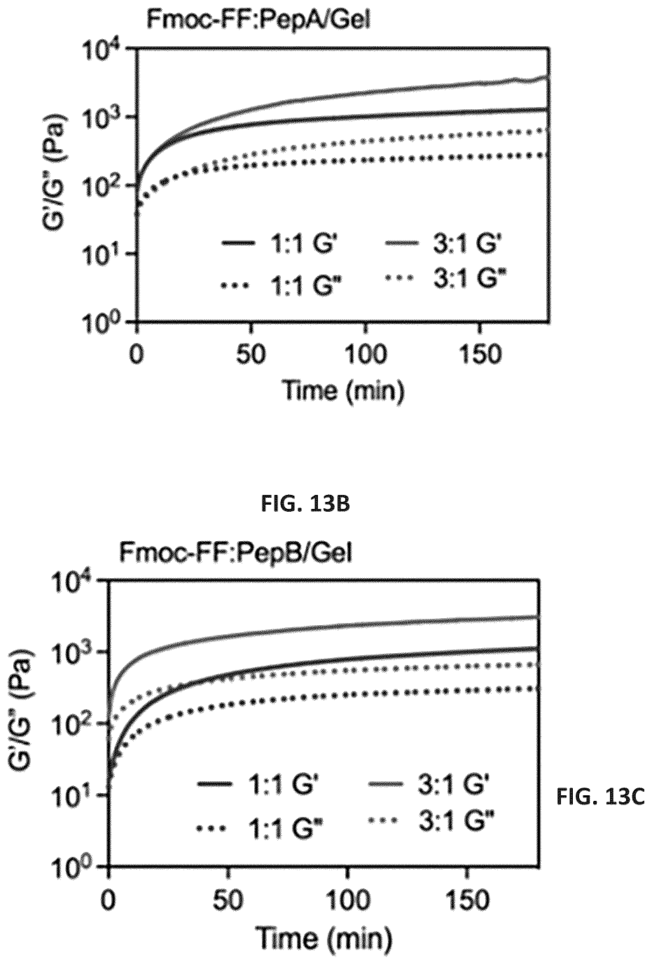

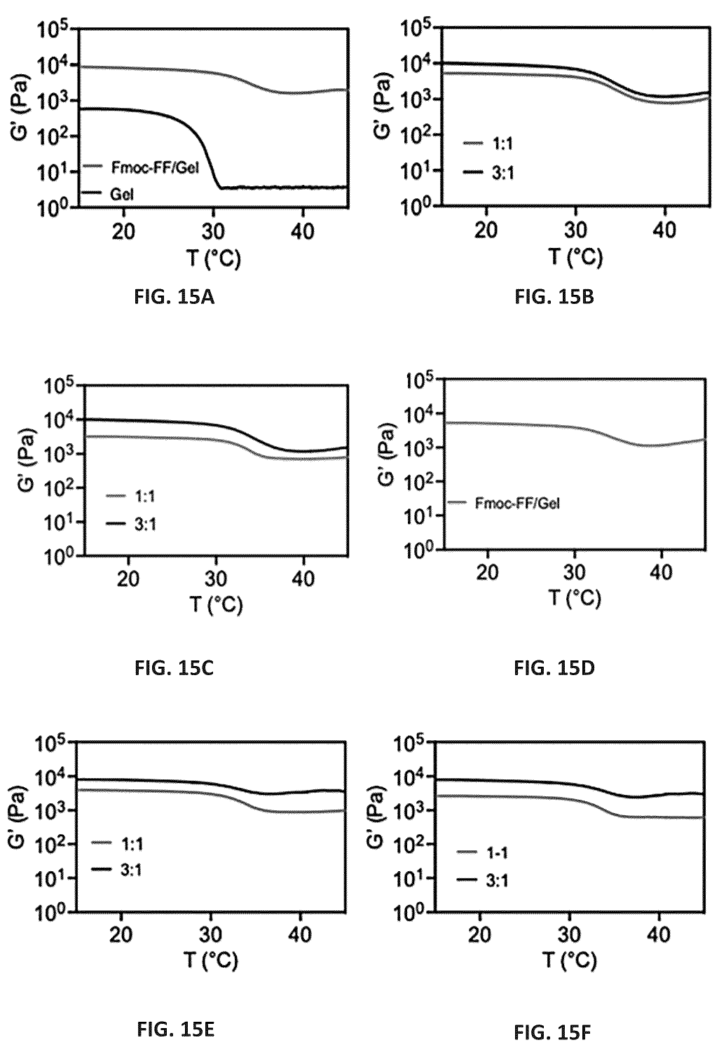

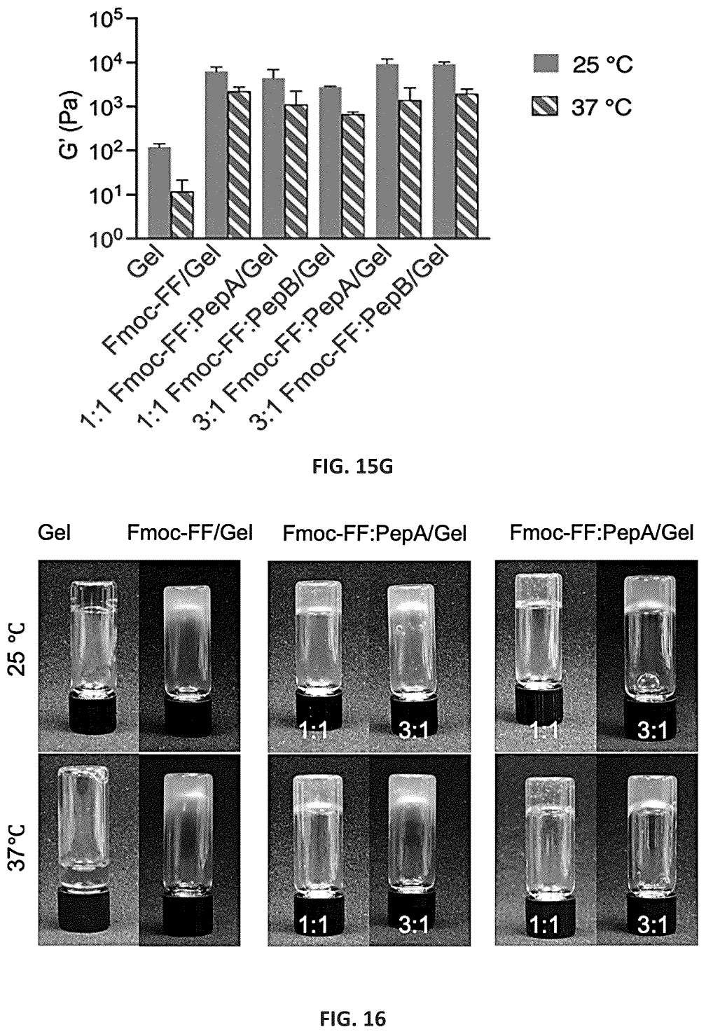

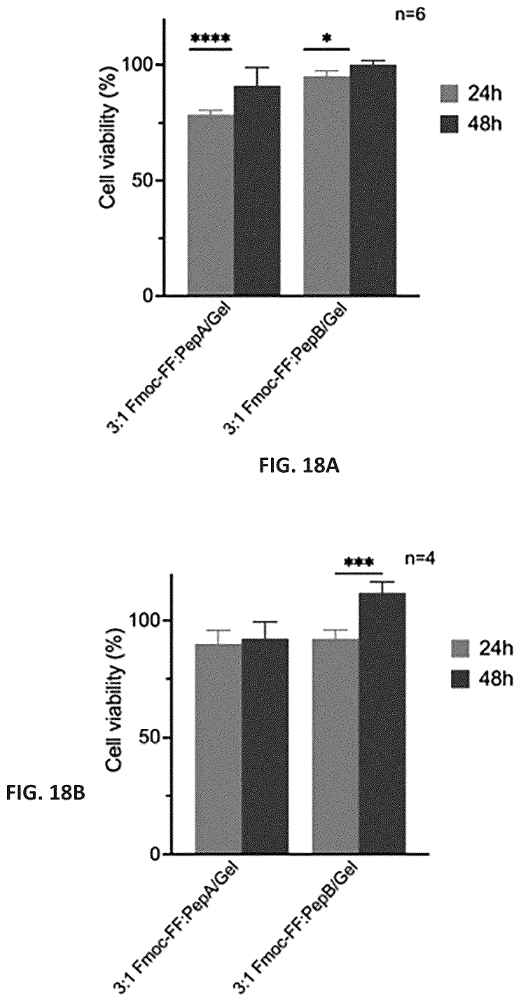

The present inventors have demonstrated that combining gelatin, as an exemplary matrix-forming material, with the self-assembling peptide-based formulations, provides composite hydrogels and composite hybrid hydrogels that meet the bioprinting process requirements and can be successfully used as bioink compositions, in terms of injectability (see, FIGS. 11A-B), printability (see, FIGS. 12A-C and 17A-D), mechanical and rheological properties of the bioink compositions (see, FIGS. 13A-C) and thermostability (see, FIGS. 15A-G and 16). The biocompatibility of objects printed using such bioink compositions has been demonstrated (FIGS. 18A-B, 19 and 20A-B). Thus, the use of bioink compositions that combine a matrix-forming material with self-assembled peptide-based (optionally hybrid) hydrogels has been successfully practiced and represents a new approach in bioprinting, by enabling fabrications of biocompatible objects without the need for any post-printing procedures.

Embodiments of the present invention relate to a newly designed formulation that comprises a plurality of self-assembling peptides, to a newly designed bioink composition prepared by combining self-assembled peptide-based hydrogels or hybrid hydrogels with a matrix-forming material, and to additive manufacturing of three-dimensional objects using such a bioink composition. The formulations and bioink compositions can be used, inter alia, in 3D-bioprinting in the field of tissue engineering.

The bioink compositions according to at least some of the present embodiments are used in bioprinting in a form of a hydrogel, as described and defined herein.

Herein throughout, a formulation, or a hydrogel-forming formulation, is used to describe an aqueous solution that is capable of forming a hydrogel. In some embodiments, the bioink composition is in a form of a hydrogel or is capable of forming a hydrogel upon contacting an aqueous solution.

In some of any of the embodiments described herein, the formulation comprises a plurality of aromatic peptides as described herein in any of the respective embodiments that are capable of self-assembling in an aqueous solution to thereby form a hydrogel. A hydrogel according to some of the present embodiments is therefore also referred to as self-assembled hydrogel or self-assembled peptide (or peptide-based) hydrogel (or hybrid hydrogel).

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional solid-like fibrous network.

As used herein the phrase “fibrous network” refers to a set of connections formed between a plurality of fibrous components.

Herein, the fibrous components are composed, at least in part, of peptide fibrils, each formed upon self-assembly of aromatic peptides as described herein in any of the respective embodiments.

A hydrogel, according to some embodiments of the present invention, may contain macromolecular polymeric and/or fibrous elements which are not chemically connected to the main fibrous network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Such materials are also referred to herein as “matrix-forming materials”. Non-limiting examples of such macromolecules include polycaprolactone, gelatin, gelatin methacrylate, alginate, alginate methacrylate, chitosan, chitosan methacrylate, glycol chitosan, glycol chitosan methacrylate, hyaluronic acid (HA), HA methacrylate, and other non-crosslinked natural or synthetic polymeric chains and the likes. Alternatively, or in addition, such macromolecules are chemically connected to the main crosslinked network of the hydrogel, for example, by acting as a cross-linking agent, or by otherwise forming a part of the three-dimensional network of the hydrogel.

According to some of any of the embodiments described herein, the matrix-forming material is a non-curable material, namely, is devoid of curable groups that are capable of undergoing polymerization and/or cross-linking when exposed a curing condition, as described herein.

As used herein and in the art, the term “hardening” describes a process in which a formulation is hardened. The hardening of a formulation typically involves an increase in a viscosity of the formulation and/or an increase in a storage modulus of the formulation (G′). In the context of additive manufacturing (e.g., 3D bioprinting), a modeling material formulation such as a bioink composition, which is dispensed as a liquid, becomes solid or semi-solid (e.g., gel) when hardened. A formulation which is dispensed as a semi-solid (e.g., soft gel) becomes solid or a harder or stronger semi-solid (e.g., strong gel) when hardened.

In the context of additive manufacturing (e.g., 3D bioprinting), currently practiced methodologies typically used bioink compositions (as modeling material formulations) that upon being dispensed, undergo curing, and are thereby hardened.

The term “curing” as used herein and in the art encompasses, for example, polymerization of monomeric and/or oligomeric materials and/or cross-linking of polymeric chains (either of a polymer present before curing or of a polymeric material formed in a polymerization of the monomers or oligomers). The product of a curing reaction is therefore typically a polymeric material and/or a cross-linked material. This term, as used herein, encompasses also partial curing, for example, curing of at least 20% or at least 30% or at least 40% or at least 50% or at least 60% or at least 70% of the formulation, as well as 100% of the formulation.

Herein, the phrase “a condition that affects curing” or “a condition for inducing curing”, which is also referred to herein interchangeably as “curing condition” or “curing inducing condition” describes a condition which, when applied to a dispensed modeling material formulation that contains a curable material, induces a curing as defined herein. Such a condition can include, for example, application of a curing energy to the curable material(s), and/or contacting the curable material(s) with chemically reactive components such as catalysts, co-catalysts, and activators.

When a condition that induces curing comprises application of a curing energy, the phrase “exposing to a curing condition” and grammatical diversions thereof means that the dispensed layers are exposed to the curing energy and the exposure is typically performed by applying a curing energy to the dispensed layers.

A “curing energy” typically includes application of radiation or application of heat.

A matrix-forming material according to some of the present invention, when included in a bioink composition as described herein, does not undergo hardening (e.g., a change in viscosity and/or storage modulus) when exposed to a curing condition as described herein. Exemplary such matric-forming agents include, but are not limited to, polycaprolactone, gelatin, alginate, chitosan, glycol chitosan, hyaluronic acid (HA), and other non-crosslinked natural or synthetic polymeric chains and the likes. Additional examples are provided hereinunder.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymer chains, the “degree of cross-linking” (number of interconnected links between the chains), the aqueous media content and composition, and temperature.

Herein, the self-assembled peptide-based hydrogel according to the present embodiments, encompasses hydrogels formed of a plurality of aromatic peptides that are substantially the same, hydrogels formed of two or more types of aromatic peptides (also referred to herein as “hybrid hydrogels”), hydrogels formed of a plurality of aromatic peptides that are substantially the same and a matrix-forming material (also referred to herein as “composite hydrogels”), and hydrogels formed of two or more types of aromatic peptides and a matrix-forming material (also referred to herein as “composite hybrid hydrogels”).

According to some embodiments, the hydrogel is a physical hydrogel, in which the interactions between the components (e.g., self-assembled aromatic peptides and optionally a matrix-forming material) does not involve covalent bonds, such that these components are not covalently attached to one another.

As used herein, the phrase “self-assembled aromatic peptides” or “self-assembled hydrogel” made of a plurality of aromatic peptides, encompasses any aromatic peptides as described herein in any of the embodiments, that under certain conditions (e.g., concentration and/or temperature), spontaneously rearrange in an aqueous solution so as to form peptide fibrils that form the hydrogel's fibrous network in the solution. According to some embodiments of the invention, the plurality of aromatic peptides as described herein self-assemble in an aqueous solution to form a hydrogel at room temperature. According to some embodiments of the present invention, the plurality of aromatic peptides as described herein self-assemble in an aqueous solution to form a hydrogel at a concentration range of at least 1 mg/ml, or at least 2 mg/ml, or at least 2.5 mg/ml, preferably at least 3 mg/ml, preferably at least 5 mg/ml, and up to 100 mg/ml, or up to 50 mg/ml, or up to 20 mg/ml, or up to 10 mg/ml, including any intermediate values and subranges therebetween.

In some of any of the embodiments described herein, the mechanical properties and/or the stability of the hydrogel is controllable by the ratio between the first and the second portions of the plurality of peptides as described herein in any of the embodiments (e.g., a higher amount of the first portion of the plurality of peptides as described herein may provide higher G′, as described in the Example section that follows).

According to an aspect of some embodiments of the present invention, there is provided a bioink composition comprising a plurality of aromatic peptides. In some of any of the embodiments described herein, the plurality of aromatic peptides comprises at least a first portion that comprises aromatic peptides of 2 to 6 amino acid residues. In some of any of the embodiments described herein, the at least first portion comprises aromatic peptides of 2 to 6 amino acid residues features an aromatic end-capping moiety. In some of any of the embodiments described herein, the plurality of peptides further comprises at least a second portion that comprises aromatic peptides of 2 to 6 amino acid residues featuring an aromatic end-capping moiety and having a an alkylene glycol-containing moiety conjugated thereto.

### Formulation:

A formulation as described herein comprises a plurality of aromatic peptides, as described herein in any of the respective embodiments and any combination thereof, and is capable of forming a hydrogel when contacting an aqueous solution or is in a form a hydrogel, as described herein.

According to some of any of the embodiments described herein, the plurality of aromatic peptides forms a hydrogel in an aqueous solution upon self-assembly of the plurality of peptides to thereby form a fibrous network as described herein. The hydrogel formation, is some embodiments, occurs at room temperature. In some embodiments, the hydrogel formation occurs within a time period that ranges from 1 minute to several hours (e.g., 1, 2, 3, 4, 5, 6 hours, or more), after the plurality of aromatic peptides are contacted with the aqueous solution.

According to some embodiments of the present invention, the formulation comprises a plurality of peptides, at least a portion, or each, of the peptides, being aromatic peptides, as described herein. According to some embodiments, each of the peptides is of 2 to 6 amino acid residues. According to some embodiments, each of the aromatic peptides is of 2 to 6 amino acid residues.

Herein throughout, by “at least a portion” it is meant at least 10%, or at least 20%, or at least 30%, or at least 40%, or at least 50%, or at least 60%, or at least 70%, or at least 80%, or at least 90%, or all, of the indicated peptides.

In some embodiments, each peptide in the plurality of peptides independently has at least two amino acid residues and up to 6 amino acid residues.

Herein, the phrase “aromatic peptide” describes a peptide as described herein (e.g., of from 2 to 6 amino acid residues), in which at least one amino acid residue is an aromatic amino acid residue, as described herein. An aromatic peptide according to embodiments of the present invention can comprise one, two, three, four, five or six amino acid residues, with one, two, three, four, five or six aromatic amino acid residues as described herein.

According to some embodiments, each peptide in the plurality of peptides is an aromatic peptide, and comprises at least one aromatic amino acid residue.

In some embodiments of the present invention, at least one peptide in the plurality of peptides is a polyaromatic peptide, comprising two or more aromatic amino acid residues.

Thus, for example, the plurality of peptides can include any combination of: dipeptides composed of one or two aromatic amino acid residues; tripeptides including one, two or three aromatic amino acid residues; tetrapeptides including two, three or four aromatic amino acid residues; pentapeptides including two, three, four or five aromatic amino acid residues; and hexapeptides including two, three, four, five or six aromatic amino acid residues.

In some embodiments, one or more peptides in the plurality of peptides include two amino acid residues, and hence is a dipeptide.

In some embodiments, each of the peptides in the plurality of peptides comprises two amino acid residues such that the plurality of peptides essentially consists of a plurality of dipeptides.

In some embodiments, at least a portion (e.g., at least 50%, or at least 60%, or at least 70%, or at least 80%, or at least 90%, or at least 95%, or at least 98%, or at least 99%) or all, of the peptides in the plurality of peptides are aromatic dipeptides, namely, are peptides of 2 amino acid residues, at least one of the amino acid residues being an aromatic amino acid residue as defined herein.

In some embodiments, each peptide in the plurality of peptides is an aromatic dipeptide.

Herein, an aromatic dipeptide describes a peptide composed of two amino acid residues, wherein at least one of these amino acid residues is an aromatic amino acid residue.

The aromatic dipeptides according to any of these embodiments can be the same or different (e.g., the plurality of peptides comprises two or more types of chemically-distinct aromatic dipeptides). When the aromatic dipeptides are different, they can differ from one another by the type of a non-aromatic amino acid residue and/or by the type of the one or two aromatic amino acid residues and/or by the type of the terminal groups (e.g., end-capping moieties).

In some of any of the embodiments of the present invention, at least one peptide in the plurality of peptides is an aromatic dipeptide, comprising two aromatic amino acid residues. In some embodiments, each peptide in the plurality of peptides is an aromatic dipeptide, comprising two aromatic amino acid residues, which can be the same or different.

Thus, the plurality of peptides can be or comprise a plurality of dipeptides composed of one or two aromatic amino acid residues.

The aromatic amino acid residues composing the dipeptide can be the same, such that the dipeptide is a homodipeptide, or different. In some embodiments, the plurality of peptides comprises or consists of a plurality of aromatic homodipeptides, which comprise two aromatic amino acid residues which are the same in terms of the aromatic moiety in the side-chain thereof.

The phrase “aromatic amino acid residue”, as used herein, refers to an amino acid residue that has an aromatic moiety in its side-chain.

As used herein, the phrase “aromatic moiety” describes a monocyclic or polycyclic moiety having a completely conjugated pi-electron system. The aromatic moiety can be an all-carbon moiety or can include one or more heteroatoms such as, for example, nitrogen, sulfur or oxygen. The aromatic moiety can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine.

Exemplary aromatic moieties include, but are not limited to, phenyl, biphenyl, naphthalenyl, phenanthrenyl, anthracenyl, [1,10]phenanthrolinyl, indoles, thiophenes, thiazoles and, [2,2′]bipyridinyl, each being optionally substituted. Thus, representative examples of aromatic moieties that can serve as the side chain within the aromatic amino acid residues described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

Exemplary aromatic homodipeptide include, but are not limited to, phenylalanine-phenylalanine dipeptide (diphenylalanine peptide), naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide.

In some of any of the embodiments described herein, the plurality of aromatic dipeptides comprises a plurality of diphenylalanine peptides. In some embodiments, the plurality of aromatic dipeptides consists of diphenylalanine peptides (Phe-Phe, or FF, dipeptides).

In a preferred embodiment of the present invention, each peptide in the plurality of peptides is a homodipeptide composed of two aromatic amino acid residues that are identical with respect to their side-chain residues.

The plurality of peptides can comprise linear and/or cyclic peptides (e.g., cyclic di-peptides of phenylalanine). In exemplary embodiments, the plurality of peptides consists of linear peptides as described herein.

According to some of any of the embodiments described herein, at least a portion (as defined herein) or each, of the aromatic peptides in the plurality of peptides is an end-capping modified aromatic peptide.

According to some embodiments of the present invention, one or more, or each, of the peptides in the plurality of peptides is an end-capping modified peptide.

The phrase “end-capping modified peptide”, as used herein, refers to a peptide which has been modified at the N-(amine) terminus and/or at the C-(carboxyl) terminus thereof. The end-capping modification refers to the attachment of a chemical moiety to the terminus, so as to form a cap. Such a chemical moiety is referred to herein as an end-capping moiety and is typically also referred to herein and in the art, interchangeably, as a peptide protecting moiety or group.

The phrase “end-capping moiety”, as used herein, refers to a moiety that when attached to the terminus of the peptide, modifies the end-capping. The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2nd ed. 1991) and Harrison et al., “Compendium of Synthetic Organic Methods”, Vols. 1-8 (John Wiley and Sons, 1971-1996).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denoted herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”).

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus end-capping may be modified to a substituted or unsubstituted amide group.

Other end-capping modifications of peptides include replacement of the amine and/or carboxyl with a different moiety, such as hydroxyl, thiol, halide, alkyl, aryl, alkoxy, aryloxy and the like, as these terms are defined herein.

In a preferred embodiment of the present invention, all of the aromatic peptides are end-capping modified at the N-terminus.

End-capping moieties can be classified by their aromaticity. Thus, end-capping moieties can be aromatic or non-aromatic.

Representative examples of non-aromatic end capping moieties suitable for N-terminus modification include, without limitation, formyl, acetyl trifluoroacetyl, tert-butoxycarbonyl, trimethylsilyl, and 2-trimethylsilyl-ethanesulfonyl. Representative examples of non-aromatic end capping moieties suitable for C-terminus modification include, without limitation, amides, allyloxycarbonyl, trialkylsilyl ethers and allyl ethers.

Representative examples of aromatic end capping moieties suitable for N-terminus modification include, without limitation, fluorenylmethyloxycarbonyl (Fmoc). Representative examples of aromatic end capping moieties suitable for C-terminus modification include, without limitation, benzyl, benzyloxycarbonyl (Cbz), trityl and substituted trityl groups.

In some embodiments of the present invention, the end-capping modified peptides are N-terminus-modified peptides, modified by an aromatic (e.g. Fmoc) end-capping moiety.

The end-capping modified peptides utilized according to the present embodiments can be collectively represented by the following general Formula Ia:

R₁-[A₁]-[A₂]- . . . [Aₙ]—R₂ Formula Ia
wherein:
n is an integer from 2 to 6, preferably 2;
A₁, A₂, . . . , Aₙ are each independently an amino acid residue as this term is defined herein, provided that at least one of A₁, A₂, . . . , Aₙ is an aromatic amino acid residue as this term is defined herein;
R₁ is an N-terminus end-capping moiety or absent; and
R₂ is a C-terminus end-capping moiety or absent.

In some embodiments, R₁ is an N-terminus end-capping moiety, preferably an aromatic end-capping moiety, and R₂ is absent—that is, the peptide has at the C-terminus the carboxylic acid of the An amino acid residue.

As described hereinabove, according to some embodiments of the present invention, the plurality of peptides comprises one or more end-capping modified homodipeptide.

Representative examples of end-capping modified homodipeptides include, without limitation, an end-capping modified naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide, phenylalanine-phenylalanine dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide, whereby these homodipeptides are preferably end-capping modified by an aromatic moiety, and more preferably, are end-capping modified at the N-terminus thereof by an aromatic moiety such as Fmoc.

In some embodiments of the present invention, the plurality of peptides comprises, or consists essentially of, a plurality of dipeptides, each comprising at least one aromatic amino acid residue.

In some embodiments, in each of the dipeptides both amino acid residues are aromatic amino acid residues, as described herein.

In some embodiments, in each of the dipeptides, the aromatic amino acid residues are the same, such that each of the dipeptides is a homodipeptide, or an aromatic homodipeptide.

In some embodiments, the plurality of dipeptides comprises, or consists essentially of, a plurality of Phe-Phe homodipeptides.

In some embodiments, the plurality of dipeptides comprises, or consists essentially of, a plurality of homodipeptides (e.g., Phe-Phe) which are end-capping modified peptides, wherein the end-capping moiety in such peptides in an aromatic moiety such as Fmoc.

In some embodiments, the plurality of peptides comprises, or consists essentially of, a plurality of Fmoc-Phe-Phe (Fmoc-FF).

The plurality of peptides in a formulation as described herein can comprise one or more types of aromatic peptides (e.g., a plurality of end-capping modified aromatic peptides). According to some of any of the embodiments described herein, the plurality of peptides comprises at least two types of aromatic peptides.

According to some of any of the embodiments described herein, the plurality of peptides as described herein, whether comprising one or two or more types of peptides, is such that the plurality of peptides are capable of self-assembling an in aqueous solution to thereby form a hydrogel or a hybrid hydrogel, as described herein.

According to an aspect of some embodiments of the present invention, there is provided a formulation comprising a plurality of peptides, the plurality of peptides comprising two or more portions of peptides. According to some embodiments, one portion of the peptides (a first portion) comprises a plurality of aromatic peptides of 2 to 6 amino acid residues featuring an aromatic end-capping moiety, as described herein in any of the respective embodiments and any combination thereof, and another portion of the peptides (a second portion) comprises aromatic peptides of 2 to 6 amino acid residues featuring an aromatic end-capping moiety and having an alkylene glycol-containing moiety conjugated thereto. The plurality of peptides may further comprise a third, fourth and so forth portions of peptides, in which the peptides are different from the peptides defined for the first and second portions as described herein. These third, fourth and so forth portions can each comprise a plurality of aromatic peptides (e.g., end-capping modified aromatic peptides), which have different number of amino acid residues, different aromatic amino acid residue(s), a different alkylene glycol moiety and/or a different end-capping moiety, compared to other portions of peptides in the plurality of peptides.

According to some embodiments, the plurality of peptides (e.g., comprising two, three or more portions of different peptides) comprises peptides that are capable of self-assembling when contacting an aqueous solution, to thereby form a hydrogel, as described herein.

According to some of any of the embodiments of the present invention, the second portion that comprises aromatic peptides of 2 to 6 amino acid residues features an aromatic end-capping moiety, as described herein in any of the embodiments. According to some of any of the embodiments described herein, the second portion that comprises aromatic peptides of 2 to 6 amino acid residues have an alkylene glycol-containing moiety conjugated thereto.

As used herein, the term “alkylene glycol” describes a —[(CR′R″)_{z}—O]_{y}— group, with R′, R″ and R″′ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-10, the alkylene glycol is referred to herein as oligo(alkylene glycol).

When y is greater than 10, the alkylene glycol is referred to herein as poly(alkylene glycol).

In some embodiments, the alkylene glycol-containing moiety has from 1 to 20 repeating alkylene glycol units, such that z is 1 to 20, preferably 2-20, more preferably 4-20, and is, for example, from 2 to 12, or from 2 to 10, or from 4 to 12, or from 4 to 10, or from 6 to 12, or from 6 to 10, or from 2 to 8, or from 4 to 8, or from 2 to 6, or from 4 to 6, repeating alkylene glycol units, including any intermediate values and subranges therebetween. In some of these embodiments, each of the alkylene glycol units is an ethylene glycol unit.

As used herein, the term “alkylene glycol” encompasses any alkylene glycol, oligo(alkylene glycol), poly(alkylene glycol), cycloalkyl diol or unsaturated aliphatic diol.

The phrase “alkylene glycol-containing moiety” as used herein describes a moiety which is or comprises an alkylene glycol as described herein.

In some of any of the embodiments described herein, the alkylene glycol-containing moiety comprises an oligo(alkylene glycol) moiety. In such embodiments, the oligo(alkylene glycol) moiety comprises at least 2, and up to 6, or up to 8, or up to 10, or up to 12, or up to 14, or up to 16, or up to 18, or up to 20, repeating units of alkylene glycol. In preferred embodiments, the alkylene glycol-containing moiety comprises an oligo(alkylene glycol) moiety of 2 to 6 repeating units of alkylene glycol in its backbone.

In some of any of the embodiments described herein, the alkylene glycol-containing moiety is or comprises ethylene glycol. In an exemplary embodiment, the alkylene glycol-containing moiety is an oligo(ethylene glycol) moiety of 2 to 6 repeating units of ethylene glycol.

In some of any of the embodiments described herein, the alkylene glycol-containing moiety is represented by the formula: —X—[(CR′R″)z-O]y-Y, wherein X is a linking group, that links the moiety to a respective group of the peptide to which it is conjugated to, and Y is an end group.

X can be, for example, —O—, —NH—, —S—, alkyl, alkoxy, and any other linking group as described herein.

Y can be, for example, hydrogen, alkyl, cycloalkyl, and is preferably a hydrophilic group, for example, an alkyl (lower) substituted by one or more of hydroxy, thiol, carboxy, alkoxy, thioalkoxy, amine, etc.

Exemplary alkylene glycol-containing moieties are presented in FIG. 1 and in the Examples section that follows.

In some of any of the embodiments described herein, the alkylene glycol-containing moiety conjugated to the C-terminus of a peptide (e.g., an N-terminus end-capping modified aromatic homodipeptide as described herein) comprising same.

In some embodiments, the alkylene glycol-containing moiety is conjugated to the peptide, e.g., to the C-terminus of the peptide, by coupling a respective amine derivative of the alkylene glycol-containing moiety to a chemically compatible group of the peptide (e.g., the terminal carboxylic acid). Such an amine derivative is —NH—[(CR′R″)z-O]y-Y.

As used herein, the phrase “conjugated thereto” describes the chemical conjugation of the alkylene glycol-containing moiety, as described herein, to the aromatic peptides that feature an aromatic end-capping moiety, as described herein, such that the moiety is covalently bound to the aromatic peptide, to thereby provide an alkylene glycol-modified aromatic peptide.

In some of any of the embodiments described herein, the aromatic peptides that feature an aromatic end-capping moiety are conjugated to the alkylene glycol-containing moiety at the C-terminus.

In a preferred embodiment, the aromatic peptides feature an aromatic end-capping moiety that are end-capping modified only at the N-termini, and are conjugated to the alkylene glycol-containing moiety at the C-terminus. However, other combinations of N-terminus and C-terminus end-capping and alkylene glycol-containing moiety conjugated of the various aromatic peptides composing the bioink composition are also contemplated. These include, for example, the presence of certain percent of end-capping modified and alkylene glycol-containing moiety conjugated aromatic peptides within the plurality of peptides, whereby the aromatic peptides are modified and/or conjugated at the N-termini and/or the C-termini.

The end-capping modified peptides having an alkylene glycol-containing moiety conjugated to the C-terminus utilized according to the present embodiments can be collectively represented by the following general Formula Ib:

R₁-[A₁]-[A₂]- . . . [Aₙ]—R₂ Formula Ib
wherein:
n is an integer from 2 to 6, preferably 2;
A₁, A₂, . . . , Aₙ are each independently an amino acid residue as this term is defined herein, provided that at least one of A₁, A₂, . . . , Aₙ is an aromatic amino acid residue as this term is defined herein;
R₁ is an N-terminus end-capping moiety as described herein in any of the respective embodiments; and
R₂ is an alkylene glycol-containing moiety as described herein in any of the respective embodiments.

In some embodiments, R₁ in an N-terminus aromatic end-capping moiety as described herein in any of the respective embodiments.

In some of any of these embodiments, and R₂ is —NH—[(CR′R″)z-O]y-Y, as described herein. In some of these embodiments, Y is or comprises a carboxylate, preferably a carboxylic acid group, or any other hydrophilic group, for example, a —(CH₂)q-C(═O)OR″′ group, wherein q is an integer of from 1 to 6, or 1 to 4, or 1 or 2, and R″′ is as defined herein and in preferably hydrogen. According to some of any of the embodiments described herein, a weight ratio between the first, second, and other portions of the plurality of peptides, if present, can be selected, tuned or determined, to thereby control a property (e.g., mechanical, chemical, rheological and/or chemical property) of a hybrid hydrogel or a composite hybrid hydrogel formed thereof.

According to some of any of the embodiments described herein, a weight ratio of the first and second portions in the plurality of peptides, as described herein, ranges from 10:1 to 1:10 or from 5:1 to 1:5, or from 3:1 to 1:3, or from 3:1 to 1:1, including any intermediate values and subranges therebetween.

According to some of any of the embodiments described herein, there is provided a formulation comprising a plurality of peptides which comprises at least a first portion comprising aromatic peptides of 2 to 6 amino acid residues featuring an aromatic end-capping moiety, as described herein in any of the respective embodiments. According to some embodiments, the formulation further comprises at least a second portion comprising aromatic peptides of 2 to 6 amino acid residues featuring an aromatic end-capping moiety and having an alkylene glycol-containing moiety conjugated thereto, as described herein in any of the respective embodiments. According to some of any of these embodiments, the plurality of peptides are capable of self-assembling to thereby form a hydrogel which contacting an aqueous solution.

According to some of any of these embodiments, a mechanical property of the hydrogel is controllable by selecting a weight ratio of the first and second portions.

According to some embodiments, a formulation as described here is usable is usable, or is for use, in preparing an ink composition (e.g., a bioink composition), when contacted with an aqueous solution to thereby form a hydrogel.

According to some embodiments, an ink or bioink composition is prepared from the formulation by contacting it with the matrix-forming agent, for example, with an aqueous solution that comprises the matrix-forming agent. According to some embodiments, a weight ratio of a first, second and optionally more, portions of the plurality of peptides in the formulation is selected in accordance with desired properties of the ink composition.

According to some of any of the embodiments described herein, the formulation further comprises a matrix-forming material, as described herein in any of the respective embodiments.

According to some embodiments, the matrix-forming material is a biocompatible material, and the formulation is usable, or is for use, in preparing a bioink composition.

According to an aspect of some embodiments of the present invention, there is provided a method of preparing a bioink composition, the method comprising contacting a formulation comprising a plurality of aromatic peptides as described herein in any of the respective embodiments and any combination thereof with an aqueous solution. According to some embodiments, the contacting is effected under conditions that enable or promote self-assembling of the plurality of aromatic peptides, to thereby form a hydrogel.

According to some of any of the embodiments described herein, the contacting is such that a final concentration of the peptides in the aqueous solution is of at least 1 mg/ml, or at least 2 mg/ml, or at least 2.5 mg/ml, preferably at least 3 mg/ml, preferably at least 5 mg/ml, and up to 100 mg/ml, or up to 50 mg/ml, or up to 20 mg/ml, or up to 10 mg/ml, including any intermediate values and subranges therebetween.

According to some of the embodiments of this aspect of the present invention, the plurality of aromatic peptides comprises at least two portions of peptides that differ from one another, that is, at least one first portion and at least one second portion of peptides, as described herein in any of the respective embodiments.

Accordingly, preparing the ink (e.g., bioink) composition can further comprise preparing a formulation as described herein, by selecting a desirable ratio of at least first and second portions of the peptides, and contacting the obtained formulation with an aqueous solution and/or with the matrix-forming agent (e.g., an aqueous solution comprising same).

In some embodiments, the plurality of peptides is dissolved in a water-miscible organic solvent, prior to contacting the plurality of peptides with the aqueous solution.

Contacting a solution of the peptides dissolved in an organic solvent as described herein with an aqueous solution can be regarded as diluting the peptide's solution to a concentration that allows self-assembly of the peptides.

The phrase “water-miscible organic solvent”, as used herein, refers to organic solvents that are soluble or miscible in water (e.g., when mixed with water at equal volumes at room temperature). Several factors inherent in the structure of the solvent molecules can affect the miscibility of organic solvents in water, such as for example, the length of the carbon chain and the type of functional groups therein. Hydrogen bonding plays a key role in making organic solvents miscible in water. For example, in alcohols, the hydroxyl group can form hydrogen bonding with water molecules. In addition, aldehydes, ketones and carboxylic acids can form hydrogen bonding via the carbonyl oxygen. Hydrogen bonding between ether and water molecules is also possible, enabling some degree of miscibility of simple ethers in water.

Examples of water-miscible organic solvents include, without limitation, simple alcohols, such as, methanol, ethanol, 1-propanol, 2-propanol, 1-butanol, 2-butanol, 2-methyl-1-propanol, 2-methyl-2-propanol, 2,2-dimethyl-1-propanol and their halogen substituted analogues, ethylene glycol, acetone, dimethylsulfoxide, acetic acid diethyl ether, tetrahydrofuran etc.

Representative examples of organic solvents that were successfully practiced in generating exemplary hydrogels according to the present invention include, acetone, dimethylsulfoxide and hexafluoroisopropanol (e.g., 1,1,1,3,3,3-hexafluoro-2-propanol, abbreviated herein as HFIP).

In some embodiments, the organic solvent is dimethylsulfoxide (abbreviated DMSO).

In some of any of the embodiments described herein, the formulation as described herein further comprises the plurality of peptides as described in any of the respective embodiments, dissolved in the water-miscible organic solvent. Such a formulation is also referred to herein as a stock solution of the peptides. A concentration of the peptides in the stock solution should preferably be higher by at least one order of magnitude than a final concentration of the peptides in the aqueous solution.

According to some of any of the embodiments of this aspect of the present invention, the ink (e.g., bioink) composition further comprises a matrix-forming material as described herein.

According to some of these embodiments, the formulation further comprises the matrix-forming material, and preparing the ink composition comprises contacting the formulation with an aqueous solution as described herein in any of the respective embodiments.

Alternatively, according to some of these embodiments, the formulation comprising the plurality of peptides is contacted with an aqueous solution that comprises the matrix-forming material (e.g., in which the matrix-forming material is dissolved).

A concentration of the matrix-forming material in the aqueous solution can be selected in accordance with a desired final concentration of this material and/or in accordance with a desired ratio of the matrix-forming material and the plurality of peptides, as described herein.

In exemplary embodiments, a concentration of the matrix-forming material in the aqueous solution ranges from 1 to 1,000, or from 1 to 500, or from 1 to 200, or from 1 to 100, or from 10 to 1,000, or from 10 to 500, or from 10 to 200, or from 10 to 100, or from 50 to 1,000, or from 50 to 500, or from 50 to 300, or from 50 to 200, or from 50 to 100, or from 100 to 1,000, or from 100 to 500, or from 100 to 300, or from 100 to 200, or from 1 to 150, or from 1 to 30, or from 1 to 20, or from 1 to 10, or from 5 to 500, or from 5 to 300, or from 5 to 200, or from 5 to 100, or from 5 to 50, mg/ml, including any intermediate values and subranges therebetween.

The final concentration of the plurality of peptide and of the biocompatible polymer in the aqueous solution, and also of the organic solvent as described herein, and hence in the generated hybrid hydrogel can be readily determined by determining the concentration of each component in the aqueous solution and/or in the hydrogel forming the ink composition.

For example, one can prepare a stock solution of the peptides in a water-miscible organic solvent or in a solvent mixture of such an organic solvent and water, in a certain concentration of the peptides, and a stock aqueous solution in which the matrix-forming material is dissolved in the same concentration of the matrix-forming material as for the peptide solution, and then mix the desired relative amounts of the solutions, so as to determine the ratio between the components in hybrid hydrogel. The concentration of each component in the solution will determine the final total concentration of the components in the composite hydrogel. Other manipulations of the concentration of each component in its stock solution and of the ratio between the stock solutions when contacted are also contemplated.

A method as described herein for preparing an ink (e.g., bioink) composition can be regarded as a method of forming a respective hydrogel. When the plurality of aromatic peptides comprises one type of peptides (e.g., a first or a second portion as described herein in any of the respective embodiments), the method is for forming a hydrogel. When the method further comprises a matrix-forming material, it is for forming a composite hydrogel. When the plurality of aromatic peptides comprises two or more types of aromatic peptides, as described herein in any of the respective embodiments, the method is for forming a hybrid hydrogel. When the method further comprises a matrix-forming material, it is for forming a composite hybrid hydrogel.

Methods of preparing bioink compositions that further comprise a biocompatible matrix-forming material such as gelatin, are considered also a methods of stabilizing the matrix-forming material and/or of facilitating its use in bioprinting, while circumventing and avoiding the need to apply post-printing steps such as application of curing energy as described herein.

The process of generating the hydrogels (including hybrid and/or composite hydrogels) described hereinabove is preferably performed at room temperature. Alternatively, it can be effected at a physiological temperature (e.g., at 37° C.).

In some embodiments, contacting is further effected by mixing the formed aqueous solution. Mixing can be performed, for example, by manual or mechanical shaking (e.g., by vortex), or by magnetic or mechanical stirring. In some embodiments, missing is performed by means of vortex.

According to an aspect of some embodiments of the present invention there is provided a kit that comprises a formulation as described herein in any of the respective embodiments, and optionally further comprises a matrix-forming agent or an aqueous solution comprising same.

In some embodiments, the formulation and the matrix-forming agent or an aqueous solution comprising same are packaged individually in the kit.

In some embodiments, the formulation is in a lyophilized or dried form, and/or comprises only the plurality of peptides, and instructions to prepare an aqueous solution of the peptides to provide a desired formulation. For example, the different portions can be individually packaged in the kit and the instructions are to prepare a formulation by selecting a weight ratio of the different portions of peptides, and preparing an aqueous solution thereof at a selected concentration. Alternatively, the kit already comprises a selected weight ratio of the peptides, either individually packaged or mixed together, and instructions to prepare an aqueous solution thereof at a selected concentration. In such embodiments, the matrix-forming agent can be an aqueous solution or also in a dry form with instructions to prepare an aqueous solution thereof.

According to some embodiments, the instructions provided in the kit for preparing an ink composition follow a method of preparing the composition, as described herein.

The kit may further comprise a water-miscible organic solvent as described herein, either individually packaged with the kit or can comprise a stock solution of the peptides in the water-miscible organic solvent, as described herein.

According to some of any of the embodiments described herein, the aqueous solvent of an aqueous solution as described herein in any of the respective embodiments, or in a hydrogel as described herein, can be water, a buffer featuring pH in a range of from about 4 to about 10, or from about 6 to about 8, or from about 7 to about 7.4, a basic aqueous solution or an acidic aqueous solution.

The aqueous solution can comprise salts and other water-soluble materials at varying concentrations.

In some embodiments, the aqueous solution comprises salts at physiologically acceptable concentrations, such that the formulation features osmolarity around a physiological osmolarity.

In some embodiments, the aqueous solution comprises a phosphate buffer and in some embodiments, the aqueous solution comprises a phosphate buffer saline, which comprises sodium phosphate monobasic and/or sodium phosphate dibasic and NaCl.

The phosphate buffer saline (PBS) can be a commercially available PBS (e.g., DPBS) or a custom-made buffer featuring a desirable pH and/or osmolarity.

Any other buffers are also usable in the context of the present embodiments.

In some of any of the embodiments described herein, the aqueous solution comprises a culturing medium. The culturing medium can be a commercially available culturing medium or a custom-made culturing medium. The culture medium can be any liquid medium that allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha′emek, Israel; Gibco-Invitrogen Corporation products, Grand Island, NY, USA).

The culture medium may be supplemented with various antibiotics (e.g., Penicillin and Streptomycin), growth factors or hormones, specific amino acids (e.g., L-glutamin) cytokines and the like.

### Bioink Composition:

According to an aspect of some embodiments of the present invention there is provided a bioink composition, comprising a matrix-forming biocompatible material and a self-assembled hydrogel formed of a plurality of peptides, wherein at least a portion of the plurality of peptides comprises aromatic peptides of 2 to 6 amino acid residues featuring an aromatic end-capping moiety. A bioink composition according to the present embodiments comprises a self-assembled hydrogel formed of a plurality of peptides such as described herein in the context of a formulation, in any of the respective embodiments and any combination thereof, and a matrix-forming material as described herein, associated with the hydrogel (e.g., being intertwined or entangled with the fibrous network formed of self-assembled peptides, as described herein).

According to some of any of the embodiments described herein, the plurality of peptides comprises at least one first portion that comprises the aromatic peptides of 2 to 6 amino acid residues featuring an aromatic end-capping moiety and at least one second portion that comprises aromatic peptides of 2 to 6 amino acid residues featuring an aromatic end-capping moiety and having an alkylene glycol-containing moiety conjugated thereto, as these are described herein in any of the respective embodiments and any combination thereof.

Depending on the type of peptides that form the self-assembled hydrogel, the bioink composition is a form of a composite hydrogel or a composite hybrid hydrogel, as described herein.

As used herein, the phrase “matrix-forming material” encompasses a material that improves the mechanical strength and/or stability of the bioink (by, e.g., increasing the viscosity of the bioink and/or increasing the storage modulus of the bioink (G′)), and/or facilitates the formation of three-dimensional fibrous network as described herein, with the plurality of peptides that form the bioink composition as described herein in any of the embodiments.

Exemplary matrix-forming materials include, but are not limited to, synthetic polymers (e.g., polyvinyl alcohol (PVA), polyethylene glycol (PEG), polyacrylamide (PAAm)), natural polymers (e.g., alginate, chitosan, hyaluronic acid), proteins (e.g., gelatin, casein) and carbohydrates (e.g., cellulose, starch).

In some of any of the embodiments described herein, the matrix-forming material is a biocompatible matrix-forming adjuvant. In a preferred embodiment, the matrix-forming adjuvant is gelatin.

As currently accepted in the art, the term “biocompatible” is generally defined as “the ability of a material to perform with an appropriate host response in a specific application” [see, The Williams dictionary of Biomaterials].

In the context of biomaterial applications such as bone regeneration, biocompatibility refers to the ability to perform as a supportive matrix to an appropriate cellular activity, without eliciting any undesirable effects in those cells, or inducing any undesirable local or systemic responses in the host.

In the context of embodiments of the present invention, a “biocompatible material” describes a material (e.g., matrix-forming material) or matrix (e.g., hydrogel or scaffold) that does not interfere, and preferably provides a suitable environment for, cellular activity.

A “cellular activity” includes, for example, cell viability, cell growth (proliferation), cell differentiation, cell migration, cell adhesion, molecular and mechanical signaling systems, and fluid transport through cells or a tissue so as to allow nutritive environment.

The biocompatibility of a substance can be determined by methods well known in the art, following the definitions hereinabove and international guidelines, using widely recognized safety assays. Optionally, biocompatible substances can be selected from existing lists of such substances.

According to some embodiments, the weight ratio between the plurality of peptides and the matrix-forming material is in the range of from 10,000:1 to 1:10,000, or from 1,000:1 to 1:1, 1000, or from 500:1 to 1:500, or from 1:100 to 100:1, or from 1:50 to 50:1 or from 20:1 to 20:1, or from 1:10 to 10:1, or from 1:5 to 5:1, or from 1:3 to 3:1, or from 1:2 to 2:1 or is about 1:1, or from 1:10,000 to 1:1, or from 1:1,000 to 1:1, or from 1:500 to 1:10, or from 1:500 to 1:50, or from 1:500 to 1:100, or from 1:500 to 1:10, including any intermediate values and subranges therebetween. In some embodiments, this weigh ratio ranges from 1:200 to 200:1, or from 1:200 to 100:1, or from 1:200 to 10:1, or from 1:200 to 1:1, or from 1:100 to 100:1, or from 1:100 to 10:1, or from 1:200 to 1:1, or from 1:50 to 50:1, or from 1:50 to 10:1, or from 1:50 to 10:1, or from 1:20 to 20:1, or from 1:20 to 10:1, or from 1:20 to 1:1, or from 1:20 to 1:10, including any intermediate values and subranges therebetween. In exemplary embodiments, the weight ratio ranges from 1:5 to 1:15.

According to some of any of the embodiments described herein, the bioink composition features a storage modulus (G′) in the range of from 1000 to 5000 Pa, or from 1000 to 4000 Pa, including any intermediate values and subranges therebetween, at 25° C., when comprising a matrix-forming adjuvant.

In some embodiments, increasing the amount of the second portion of the plurality of peptides as described herein decreases the viscosity of the bioink composition, and addition of the matrix-forming adjuvant as described herein increases the viscosity of the bioink composition.

In some embodiments, the total concentration of the plurality of peptides forming the self-assembled hydrogel and the matrix-forming material, in the composition ranges from 1 to 100, or from 1 to 50, or from 1 to 10, or from 1 to 5, or from 5 to 100, or from 5 to 50, or from 5 to 20, or from 5 to 10, weight percent of the total weight of the composition, including any intermediate values and subranges therebetween, with the balance being water or an aqueous solution (e.g., a buffer), as described herein. According to some embodiments, the bioink composition is characterized by one or more, two or more, three or more, or all of the following:
Injectability through a syringe of at least 20 gauge, determined, for example, as described in the Examples section that follows;
Printability parameter in a range of from 0.5 to 1.5, or from 0.8 to 1.2 determined, for example, as described in the Examples section that follows;
Storage modulus higher by at least 2-folds compared to a storage modulus of the matrix-forming material;
Thermostability, determined, for example, as described in the Examples section that follows; and
Biocompatibility, as defined herein.

According to some of any of the embodiments, the bioink composition further comprises at least one biological component or material, as described herein in any of the respective embodiments, other that the plurality of peptides and the matrix-forming agent as described herein.

Biological components or materials that can be included a bioink composition as described herein include, cellular components, including, for example, culturing cells, and other cellular components such as cytokines, chemokines, growth factors; as well as other biological components such as proteins, agents that act to increase cell attachment, cell spreading, cell proliferation, cell differentiation and/or cell migration; an amino acid, peptides, polypeptides, proteins, DNA, RNA, lipids and/or proteoglycans.

Cells may comprise a heterogeneous population of cells or alternatively the cells may comprise a homogeneous population of cells. Such cells can be for example stem cells (such as embryonic stem cells, bone marrow stem cells, cord blood cells, mesenchymal stem cells, adult tissue stem cells), progenitor cells, or differentiated cells such as chondrocytes, osteoblasts, connective tissue cells (e.g., fibrocytes, fibroblasts and adipose cells), endothelial and epithelial cells. The cells may be naïve or genetically modified.

According to one embodiment of this aspect of the present invention, the cells are mammalian in origin.

Furthermore, the cells may be of autologous origin or non-autologous origin, such as postpartum-derived cells (as described in U.S. application Ser. Nos. 10/887,012 and 10/887,446). Typically the cells are selected according to the desired application.

Suitable proteins which can be used include, but are not limited to, extracellular matrix proteins [e.g., fibrinogen, collagen, fibronectin, vimentin, microtubule-associated protein 1D, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth factor-α, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS17, tryptase-gamma, and matriptase-2] and protease substrates.

In addition, calcium phosphate materials, such as hydroxyapattite, for example, in a form of particles, can be used, including, but not limited to, nanoHA and nanoTCP. The particles size should be compatible with the dispensing heads so as to avoid clogging.

A bioink composition as described herein can further include one or more non-curable materials, other than the biological materials as described herein, including, for example, materials that impart a certain property to the composition. Such a property can be a physical property (e.g., an optical property such as transparency or opacity, color, a spectral property, heat resistance, electrical property and the like), or a mechanical or rheological property such as viscosity, elasticity, storage modulus, loss modulus, stiffness, hardness, and the like. Alternatively, or in addition, non-curable materials can be such that provide a biological function, for example, therapeutically active agents.

Exemplary non-curable materials include thixotropic agents, reinforcing agents, toughening agents, fillers, colorants, pigments, etc.

According to some of any of the embodiments described herein the bioink composition features a neutral pH (e.g., from about 6 to about 8).

According to some of any of the embodiments described herein the bioink composition features viscosity parameters essentially as described herein.

According to an aspect of some embodiments of the present invention, there is provided a kit that comprises a bioink composition as described herein in any of the respective embodiments.

According to some embodiments, the composition comprises the bioink composition in a lyophilized form.

For example, once the composite (optionally hybrid) hydrogel forms, the composition is dried (e.g., lyophilized), such that composition comprises the fibrous network forms of the plurality of peptides and the matrix-forming material, and kit further comprises a pharmaceutically acceptable aqueous solution, as described herein which is to be mixed with the dried hydrogel.

According to some of any of the embodiments described herein, the kit is identified for use, or is usable, as a modeling material formulation for additive manufacturing (e.g., bioprinting) of an object as described herein in any of the respective embodiments.

According to some of any of the embodiments described herein, the kit further comprises an aqueous carrier or solution, as described herein in any of the respective embodiments. In some embodiments, the composition and the aqueous carrier or solution are packaged individually within the kit.

Alternatively, the kit includes instructions to prepare a modeling material formulation as described herein, by mixing the bioink composition with the aqueous carrier or solvent, as described herein. Additive manufacturing:

According to an aspect of some embodiments of the present invention, there is provided a process (a method) of additive manufacturing (AM) of a three-dimensional object. According to embodiments of this aspect, the method is effected by sequentially forming a plurality of layers in a configured pattern corresponding to the shape of the object, thereby forming the object.

In some of any of the embodiments of this aspect, the method comprising dispensing at least one bioink composition as described herein in any of the embodiments, to sequentially form a plurality of layers in a configured pattern corresponding to a shape of the object. In some embodiments, formation of each layer is effected by dispensing at least one uncured building material, and exposing the dispensed building material to a curing condition to thereby form a hardened (cured) material.

Herein throughout, the phrase “building material” encompasses the phrases “uncured building material” or “uncured building material formulation” and collectively describes the materials that are dispensed by sequentially forming the layers, as described herein. This phrase encompasses uncured materials which form the final object, namely, one or more uncured modeling material formulation(s), and optionally also uncured materials used to form a support, namely uncured support material formulations. The building material can also include non-curable materials that do not undergo (or are not intended to undergo) any change during the process, for example, biological materials or components (other than the bioink composition as described herein) and/or other agents or additives as described herein.

The building material that is dispensed to sequentially form the layers as described herein is also referred to herein interchangeably as “printing medium” or “bioprinting medium”.

Aₙ uncured building material can comprise one or more modeling material formulations, and can be dispensed such that different parts of the object are made upon hardening (e.g., curing) of different modeling formulations, and hence are made of different hardened (e.g., cured) modeling materials or different mixtures of hardened (e.g., cured) modeling materials.

The method of the present embodiments manufactures three-dimensional object(s) in a layerwise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different bioink composition as described herein in any of the respective embodiments; or each containing a different biological component; or each containing a different curable material; or each containing a different concentration of a curable material, and/or different support material formulations). Thus, different target locations can be occupied by different building materials (e.g., a modeling formulation and/or a support formulation, as defined herein).

The final three-dimensional object is made of the hardened modeling material or a combination of hardened modeling materials or a combination of hardened modeling material/s and support material/s or modification thereof (e.g., following curing). All these operations are well-known to those skilled in the art of additive manufacturing (also known as solid freeform fabrication).

In some exemplary embodiments of the invention an object is manufactured by dispensing a building material that comprises two or more different modeling material formulations, each modeling material formulation from a different dispensing head of the AM apparatus. The modeling material formulations are optionally and preferably deposited in layers during the same pass of the dispensing heads. The modeling material formulations and/or combination of formulations within the layer are selected according to the desired properties of the object.

Aₙ exemplary process according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD).

The process continues by dispensing the building material as described herein in layers, on a receiving medium, using one or more dispensing (e.g., printing) heads, according to the printing data.

The dispensing can be in a form of droplets, or a continuous stream, depending on the additive manufacturing methodology employed and the configuration of choice.

The receiving medium can be a tray of a printing system, or a supporting article or medium made of, or coated by, a biocompatible material, such as support media or articles commonly used in bioprinting, or a previously deposited layer.

Once the uncured building material is dispensed on the receiving medium according to the 3D data, the method optionally and preferably continues by hardening the dispensed formulation(s).

In some embodiments, the hardening is performed without applying an external condition (e.g., irradiation), but is rather performed by allowing the dispensed compositions to harden, at room temperature or at an elevated temperature (e.g., up to 37 degree Celsius).

The present embodiments thus enable the deposition of a broad range of material combinations, and the fabrication of an object which may consist of multiple different combinations of modeling material formulations, in different parts of the object, according to the properties desired to characterize each part of the object.

A system utilized in additive manufacturing may include a receiving medium and one or more dispensing heads. The receiving medium can be, for example, a fabrication tray that may include a horizontal surface to carry the material dispensed from the printing head. In some embodiments, the receiving medium is made of, or coated by, a biocompatible material, as described herein.

The dispensing head may be, for example, a printing head having a plurality of dispensing nozzles arranged in an array of one or more rows along the longitudinal axis of the dispensing head. The dispensing head may be located such that its longitudinal axis is substantially parallel to the indexing direction.

The additive manufacturing system may further include a controller, such as a microprocessor to control the AM process, for example, the movement of the dispensing head according to a pre-defined scanning plan (e.g., a CAD configuration converted to a Standard Tessellation Language (STL) format and programmed into the controller). The dispensing head may include a plurality of jetting nozzles. The jetting nozzles dispense material onto the receiving medium to create the layers representing cross sections of a 3D object.

Additionally, the AM system may include a leveling device for leveling and/or establishing the height of each layer after deposition and at least partial solidification, prior to the deposition of a subsequent layer.

According to the present embodiments, the additive manufacturing method described herein is for bioprinting a biological object.

As used herein, “bioprinting” means practicing an additive manufacturing process while utilizing one or more bioink composition(s) that comprise(s) biological components, as described herein, via methodology that is compatible with an automated or semi-automated, computer-aided, additive manufacturing system as described herein (e.g., a bioprinter or a bioprinting system).

Herein throughout, the phrase “modeling material formulation”, which is also referred to herein interchangeably as “modeling formulation” or “modeling material composition” or “modeling composition”, or simply as a “formulation”, or a “composition”, describes a part or all of the uncured building material (printing medium) which is dispensed so as to form the final object, as described herein. The modeling formulation is an uncured modeling formulation, which, upon exposure to a curing condition, forms the object or a part thereof.

In the context of bioprinting, an uncured building material comprises at least one modeling formulation that comprises one or more biological components or materials, and is also referred to herein and in the art as “bioink” or “bioink formulation”.

Herein a modeling material formulation is a bioink composition as described herein in any of the respective embodiments and any combination thereof.

In some embodiments, the bioprinting comprises sequential formation of a plurality of layers of the uncured building material in a configured pattern, preferably according to a three-dimensional printing data, as described herein. At least one, and preferably most or all, of the formed layers comprise(s) one or more Bioink compositions as described herein.

In some of any of the embodiments described herein, the building material (e.g., the printing medium) comprises modeling material formulation(s) and optionally support material formulation(s), and all are selected to include materials or combination of materials that do not interfere with the biological and/or structural features of the biological components.

In some of any of the embodiments described herein, the bioprinting method is configured to effect formation of the layers under conditions that do not significantly affect structural and/or functional properties of the biological components in the bioink.

In some embodiments, a bioprinting system for effecting a bioprinting process/method as described herein is configured so as to allow formation of the layers under conditions that do not significantly affect structural and/or functional properties of the biological components in the bioink.

In some of any of the embodiments described herein, the additive manufacturing (e.g., bioprinting) process and system are configured such that the process parameters (e.g., temperature, shear forces, shear strain rate) do not interfere with (do not substantially affect) the functional and/or structural features of the biological components.

In some of any of the embodiments described herein, the additive manufacturing process (the bioprinting) is performed at a temperature of or from about 0° C. to 37° C., or from about 10° C. to 37° C., or from about 20° C. to 37° C., or from about 20° C. to about 30° C., or from about 20° C. to about 28° C., or from about 20° C. to about 25° C., including any intermediate values and subranges therebetween, or at room temperature, or at 37° C.

In some of any of the embodiments described herein, the above-indicated temperatures/temperature ranges are the temperatures at which the building material (e.g., at least a modeling material formulation that comprises a biological component as described herein) are dispensed, that is, a temperature of a dispensing head in the AM system and/or a temperature at which the modeling material formulation is maintained prior to passing in the dispensing head.

In some of any of the embodiments described herein, the additive manufacturing process (bioprinting) is performed while applying a shear force that does adversely affect structural and/or functional properties of biological components. Applying the shear force can be effected by passing the building material (e.g., at least a bioink composition that comprises a biological component as described herein) through the dispensing head, and is to be regarded also as subjecting the building material to shear force.

The following describes exemplary AM bioprinting methodologies that are usable in the context of embodiments of the present invention.

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bioink compatibility and ease-of-use.

Exemplary suitable bioprinting systems usually contain a dispensing system (either equipped with temperature control module or at ambient temperature), and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

Generally, bioprinting can be effected using any of the known techniques for additive manufacturing. The following lists some exemplary additive manufacturing techniques, although any other technique is contemplated.

### 3D Inkjet Printing:

3D Inkjet printing is a common type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received. Inkjet printers can be used with several types of ink, for example, comprising multiple types of biological components and/or bioactive agents. Furthermore, the printing is fast and can be applied onto culture plates.

A bioprinting method that utilizes a 3D inkjet printing system can be operated using one or more bioink compositions as described herein as one or more modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic or mechanical dispensing systems.

### Stereolithography (SLA) and Digital Light Processing (DLP):

SLA and DLP are additive manufacturing technologies in which an uncured building material in a bath is converted into hardened material(s), layer by layer, by selective curing using a light source while the uncured material is later separated/washed from the hardened material. SLA is widely used to create models, prototypes, patterns, and production parts for a range of industries including for Bioprinting.

### Laser-Assisted Printing:

Laser-assisted printing technique, in the version adopted for 3D bioprinting, and is based on the principle of laser-induced forward transfer (LIFT), which was developed to transfer metals and is now successfully applied to biological material. The device consists of a laser beam, a focusing system, an energy absorbing/converting layer and a biological material layer (e.g., cells and/or hydrogel) and a receiving substrate. A laser assisted printer operates by shooting a laser beam onto the absorbing layer which convert the energy into a mechanical force which drives tiny drops from the biological layer onto the substrate. A light source is then utilized to cure the material on the substrate.

Laser assisted printing is compatible with a series of viscosities and can print mammalian cells without affecting cell viability or cell function. Cells can be deposited at a density of up to 10⁸ cells/ml with microscale resolution of a single cell per drop.

### Electrospinning:

Electrospinning is a fiber production technique, which uses electric force to draw charged threads of polymer solutions, or polymer melts.

### The Object:

Herein throughout, in the context of bioprinting, the term “object” describes a final product of the additive manufacturing which comprises, in at least a portion thereof, a biological component. This term refers to the product obtained by a bioprinting method as described herein, after removal of the support material, if such has been used as part of the uncured building material.

The term “object” as used herein throughout refers to a whole object or a part thereof.

The three-dimension network or scaffold of the object can be in a form of, for example, a film, a sponge, a porous structure, a hydrogel, and any other form, according to a desired need.

In some of any of the embodiments described herein, the object is in a form of a tissue or organ, which comprises, in at least a portion thereof, a hydrogel as described herein. Such an object can be formulated in accordance with a respective 3D printing data of a desired organ or tissue, using, in addition to the aromatic peptides as described herein in any of the embodiments, curable materials and biological materials as described herein.

In some embodiments, the object is an implantable object. In some embodiments, the object is an artificial skin. In some embodiments, the object is an artificial tissue (e.g., connective tissue, or muscle tissue such as cardiac tissue and pancreatic tissue). Examples of connective tissues include, but are not limited to, cartilage (including, elastic, hyaline, and fibrocartilage), adipose tissue, reticular connective tissue, embryonic connective tissues (including mesenchymal connective tissue and mucous connective tissue), tendons, ligaments, and bone.

In some embodiments, the object is usable in, or is for use in, constructing an artificial organ or tissue.

The object can further comprise hardened materials formed of one or more of the additional curable materials as described herein in any of the respective embodiments, biological components or materials, as described herein in any of the respective embodiments, and/or non-curable materials as described herein in any of the respective embodiments.

According to an aspect of some embodiments of the present invention there is provided a three-dimensional object prepared by the additive manufacturing (bioprinting) method or process, as described herein in any of the respective embodiments.

According to an aspect of some embodiments of the present invention there is provided a three-dimensional object comprising a composition which comprises a matrix-forming biocompatible material, as described herein, and a self-assembled hydrogel formed of a plurality of peptides, the plurality of peptides comprising at least a first portion that comprises aromatic peptides of 2 to 6 amino acid residues featuring an aromatic end-capping moiety and at least a second portion that comprises aromatic peptides of 2 to 6 amino acid residues featuring an aromatic end-capping moiety and having an alkylene glycol-containing moiety conjugated thereto, as described herein, wherein the composition is shaped in a configured pattern corresponding to the shape of the three-dimensional object. Such an object can be formed by a bioprinting as described herein in any of the respective embodiments, or by any other methods known in the art for provided shaped objects (e.g., molding, sculpturing, etc.)

According to some of any of the embodiments described herein, the object, or a part thereof, comprises a hydrogel comprising a matrix-forming material and a self-assembled hydrogel as described herein.

According to some of any of the embodiments described herein, the object has a biological component as described herein associated with the composition.

In some embodiments, the object is in a form of a scaffold or film that can be used in research or therapeutic applications, for example, in repairing a damaged tissue, for example, upon seeding culturing cells therein, or in wound healing.

The scaffolds may be administered to subjects in need thereof for the regeneration of tissue such as connective tissue, muscle tissue such as cardiac tissue and pancreatic tissue.

According to some embodiments, films or scaffolds can be used in cell cultures.

The phrase “cell culture” or “culture” as used herein refers to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms.

The object of the present embodiments comprises a myriad of other uses including, but not limited to, in the treatment of diseases such as interstitial cystitis, scleroderma, and rheumatoid arthritis cosmetic surgery, as a healing aid for burn patients, as a wound-healing agent, as a dermal filler, for spinal fusion procedures, for urethral bulking, in duraplasty procedures, for reconstruction of bone and a wide variety of dental, orthopedic and surgical purposes.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Herein, the phrase “linking group” describes a group (e.g., a substituent) that is attached to two or more moieties in the compound; whereas the phrase “end group” describes a group (e.g., a substituent) that is attached to a single moiety in the compound via one atom thereof.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

The heteroalicyclic group can be an end group, as this phrase is defined herein, wherein it is attached to a single adjacent atom, or a linking group, as this phrase is defined herein, connecting two or more moieties.

Herein, the terms “amine” and “amino” each refer to either a —NR′R″ group or a —N+R′R″R″′ group, wherein R′, R″ and R″′ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R″′ are hydrogen or alkyl comprising 1 to 4 carbon atoms. Optionally, R′ and R″ (and R″′, if present) are hydrogen. When substituted, the carbon atom of an R′, R″ or R″′ hydrocarbon moiety which is bound to the nitrogen atom of the amine is not substituted by oxo (unless explicitly indicated otherwise), such that R′, R″ and R″′ are not (for example) carbonyl, C-carboxy or amide, as these groups are defined herein.

An “azide” group refers to a —N═N⁺═N⁻ end group.

An “alkoxy” group refers to any of an —O-alkyl, —O-alkenyl, —O-alkynyl, —O-cycloalkyl, and —O-heteroalicyclic end group, as defined herein, or to any of an —O-alkylene, —O-cycloalkyl- and —O-heteroalicyclic-linking group, as defined herein.

An “aryloxy” group refers to both an —O-aryl and an —O-heteroaryl group, as defined herein, or to an —O-arylene.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic end group, as defined herein, or to any of an —S-alkylene-, —S-cycloalkyl- and —S-heteroalicyclic-linking group, as defined herein.

A “thioaryloxy” group refers to both an —S-aryl and an —S-heteroaryl group, as defined herein, or to an —S-arylene.

A “carbonyl” or “acyl” group refers to a —C(═O)—R′ end group, where R′ is defined as hereinabove, or to a —C(═O)— linking group.

A “thiocarbonyl” group refers to a —C(═S)—R′ end group, where R′ is as defined herein, or to a —C(═S)— linking group.

A “carboxy”, “carboxyl”, “carboxylic” or “carboxylate” group refers to both “C-carboxy” and “O-carboxy” end groups, as defined herein, as well as to a carboxy linking group, as defined herein.

A “C-carboxy” group refers to a —C(═O)—O—R′ group, where R′ is as defined herein.

An “O-carboxy” group refers to an R′C(═O)—O—group, where R′ is as defined herein.

A “carboxy linking group” refers to a —C(═O)—O—linking group.

An “oxo” group refers to a ═O end group.

An “imine” group refers to a =N—R′ end group, where R′ is as defined herein, or to an ═N-linking group.

An “oxime” group refers to a =N—OH end group.

A “hydrazone” group refers to a =N—NR′R″ end group, where each of R′ and R″ is as defined herein, or to a=N—NR′— linking group where R′ is as defined herein.

A “halo” group refers to fluorine, chlorine, bromine or iodine.

A “sulfinyl” group refers to an —S(═O)—R′ end group, where R′ is as defined herein, or to an —S(═O)— linking group.

A “sulfonyl” group refers to an —S(═O)₂—R′ end group, where R′ is as defined herein, or to an —S(═O)₂— linking group.

A “sulfonate” group refers to an —S(═O)₂—O—R′ end group, where R′ is as defined herein, or to an —S(═O)₂—O— linking group.

A “sulfate” group refers to an —O—S(═O)₂—O—R′ end group, where R′ is as defined as herein, or to an —O—S(═O)₂—O— linking group.

A “sulfonamide” or “sulfonamido” group encompasses both S-sulfonamido and N-sulfonamido end groups, as defined herein, as well as a sulfonamide linking group, as defined herein.

An “S-sulfonamido” group refers to a —S(═O)₂—NR′R″ end group, with each of R′ and R″ as defined herein.

An “N-sulfonamido” group refers to an R'S(═O)₂—NR″— end group, where each of R′ and R″ is as defined herein.

A “sulfonamide linking group” refers to a —S(═O)₂—NR′— linking group, where R′ is as defined herein.

A “carbamyl” group encompasses both O-carbamyl and N-carbamyl end groups, as defined herein, as well as a carbamyl linking group, as defined herein.

An “O-carbamyl” group refers to an —OC(═O)—NR′R″ end group, where each of R′ and R″ is as defined herein.

An “N-carbamyl” group refers to an R′OC(═O)—NR″— end group, where each of R′ and R″ is as defined herein.

A “carbamyl linking group” refers to a —OC(═O)—NR′— linking group, where R′ is as defined herein.

A “thiocarbamyl” group encompasses O-thiocarbamyl, S-thiocarbamyl and N-thiocarbamyl end groups, as defined herein, as well as a thiocarbamyl linking group, as defined herein.

An “O-thiocarbamyl” group refers to an —OC(═S)—NR′R″ end group, where each of R′ and R″ is as defined herein.

An “N-thiocarbamyl” group refers to an R′OC(═S)NR″— end group, where each of R′ and R″ is as defined herein.

An “S-thiocarbamyl” group refers to an —SC(═O)—NR′R″ end group, where each of R′ and R″ is as defined herein.

A “thiocarbamyl linking group” refers to a —OC(═S)—NR′— or —SC(═O)—NR′— linking group, where R′ is as defined herein.

An “amide” or “amido” group encompasses C-amido and N-amido end groups, as defined herein, as well as an amide linking group, as defined herein.

A “C-amido” group refers to a —C(═O)—NR′R″ end group, where each of R′ and R″ is as defined herein.

An “N-amido” group refers to an R′C(═O)—NR″— end group, where each of R′ and R″ is as defined herein.

An “amide linking group” refers to a —C(═O)—NR′— linking group, where R′ is as defined herein.

A “urea group” refers to an —N(R′)—C(═O)—NR″R″′ end group, where each of R′, R″ and R″ is as defined herein, or an —N(R′)—C(═O)—NR″— linking group, where each of R′ and R″ is as defined herein.

A “thiourea group” refers to an —N(R′)—C(═S)—NR″R″′ end group, where each of R′, R″ and R″ is as defined herein, or an —N(R′)—C(═S)—NR″— linking group, where each of R′ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined herein, or a —P(═O)(OR′)—O— linking group, with R′ as defined herein.

The term “phosphate” describes an —O—P(═O)(OR′)(OR″) end group, with each of R′ and R″ as defined herein, or an —O—P(═O)(OR′)—O— linking group, with R′ as defined herein.

The term “phosphinyl” describes a —PR′R″ end group, with each of R′ and R″ as defined herein, or a —PR′— linking group, with R′ as defined herein.

The term “hydrazine” describes a —NR′—NR″R″′ end group, where R′, R″, and R″′ are as defined herein, or to a —NR′—NR″— linking group, where R′ and R″ are as defined herein.

As used herein, the term “hydrazide” describes a —C(═O)—NR′—NR″R″′ end group, where R′, R″ and R″′ are as defined herein, or to a —C(═O)—NR′—NR″— linking group, where R′ and R″ are as defined herein.

As used herein, the term “thiohydrazide” describes a —C(═S)—NR′—NR″R″′ end group, where R′, R″ and R″′ are as defined herein, or to a —C(═S)—NR′—NR″— linking group, where R′ and R″ are as defined herein.

A “guanidinyl” group refers to an —RaNC(═NRd)-NRbRc end group, where each of Ra, Rb, Rc and Rd can be as defined herein for R′ and R″, or to an —R′NC(═NR″)—NR″′— linking group, where R′, R″ and R″′ are as defined herein.

A “guanyl” or “guanine” group refers to an R″′R″NC(═NR′)— end group, where R′, R″ and R′ are as defined herein, or to a —R″NC(═NR′)— linking group, where R′ and R″ are as defined herein.

For any of the embodiments described herein, the compounds, materials, peptides or groups described herein may be in a form of a salt, for example, a pharmaceutically acceptable salt, and/or in a form of a prodrug.

As used herein, the phrase “pharmaceutically acceptable salt” refers to a charged species of the parent compound or material and its counter-ion, which is typically used to modify the solubility characteristics of the parent compound or material and/or to reduce any significant irritation to an organism by the parent compound or material, while not abrogating the biological activity and properties of the administered compound or material. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds, materials, peptides or groups described herein may optionally be an acid addition salt and/or a base addition salt.

Aₙ acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

Aₙ example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds, materials, peptides, groups and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

Herein, the term “peptide” refers to a polymer comprising at least 2 amino acid residues linked by peptide bonds or analogs thereof (as described herein below), and optionally only by peptide bonds per se.

The term “peptide” or “polypeptide” encompasses native peptide or polypeptide (e.g., degradation products, synthetically synthesized peptide or polypeptide and/or recombinant peptide or polypeptide), including, without limitation, native proteins, fragments of native proteins and homologs of native proteins and/or fragments thereof; as well as peptidomimetics (typically, synthetically synthesized peptides or polypeptides) and peptoids and semipeptoids which are peptides or polypeptide analogs, which may have, for example, modifications rendering the peptides or polypeptides more stable while in a body or more capable of penetrating into cells. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided herein below.

Peptide bonds (—CO—NH—) within the peptide or polypeptide may be substituted, for example, by N-methylated amide bonds (—N(CH3)—CO—), ester bonds (—C(═O)—O—), ketomethylene bonds (—CO-CH2-), sulfinylmethylene bonds (—S(═O)—CH2-), α-aza bonds (—NH—N(R)—CO—), wherein R is any alkyl (e.g., methyl), amine bonds (-CH2-NH—), sulfide bonds (-CH2-S—), ethylene bonds (-CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide or polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides or polypeptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides or polypeptides of some embodiments of the invention are preferably utilized in a linear form, although it will be appreciated that in cases where cyclization does not severely interfere with peptide or polypeptide characteristics, cyclic forms of the peptide or polypeptide can also be utilized.

Since the present peptides or polypeptides are preferably utilized in therapeutics or diagnostics which require the peptides or polypeptides to be in soluble form, the peptides or polypeptides of some embodiments of the invention preferably include one or more non-natural or natural polar amino acids, including but not limited to serine and threonine which are capable of increasing peptide or polypeptide solubility due to their hydroxyl-containing side chain.

The peptides or polypeptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide or polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide or polypeptide compounds of some embodiments of the invention involves solid phase peptide synthesis.

Large scale peptide synthesis is described by Andersson Biopolymers 2000; 55(3):227-50.

Herein, a “homolog” of a given peptide or polypeptide refers to a peptide or polypeptide that exhibits at least 80% homology, preferably at least 90% homology, and more preferably at least 95% homology, and more preferably at least 98% homology to the given peptide or polypeptide (optionally exhibiting at least 80%, at least 90% identity, at least 95%, or at least 98% sequence identity to the given peptide or polypeptide). In some embodiments, a homolog of a given peptide or polypeptide further shares a therapeutic activity with the given peptide or polypeptide. The percentage of homology refers to the percentage of amino acid residues in a first peptide or polypeptide sequence which matches a corresponding residue of a second peptide or polypeptide sequence to which the first peptide or polypeptide is being compared. Generally, the peptides or polypeptides are aligned to give maximum homology. A variety of strategies are known in the art for performing comparisons of amino acid or nucleotide sequences in order to assess degrees of identity, including, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW etc. When utilizing BLAST and Gapped BLAST programs, default parameters of the respective programs may be used. Alternatively, the practitioner may use non-default parameters depending on his or her experimental and/or other requirements (see for example, the Web site having URL www(dot)ncbi(dot)nlm(dot)nih(dot)gov).

The objects or structures of some embodiments of the invention can be administered to an organism per se, or in a pharmaceutical composition where it is mixed with suitable carriers or excipients.

As used herein a “pharmaceutical composition” refers to a preparation of one or more of the objects or structures described herein with other chemical components such as physiologically suitable carriers and excipients. The purpose of a pharmaceutical composition is to facilitate administration of objects or structures to an organism.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. Aₙ adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

As used herein, the term “subject” includes mammals, preferably human beings at any age which suffer from the pathology. Preferably, this term encompasses individuals who are at risk to develop the pathology.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Lyophilized Fmoc-Phe-Phe-OH (Fmoc-FF) was obtained from Bachem (Budendorf, Switzerland).

Lyophilized Fmoc-Phe-Phe-EG₂—COOH (PepA) and Fmoc-Phe-Phe-EG₆-COOH (PepB) were obtained from Peptron (Yuseong-daero, Republic of Korea).

Dimethyl sulfoxide (DMSO), gelatin (Gel), fluorescein and propidium iodide were obtained from Sigma-Aldrich™ (Rehovot, Israel).

AlamarBlue™ was obtained from Enco (Petach Tikvah, Israel).

Preparation of hydrogels: Stock solutions of each of Fmoc-FF, PepA and PepB were prepared by dissolving the respective peptides in dimethyl sulfoxide (DMSO) at a concentration of 100 gram/liter (g·L⁻¹). The hydrogels were prepared by diluting the stock solution in double distilled water to a final concentration of 5 grams/Liter (5 g·L⁻¹), to thereby obtain a hydrogel formulation. The mixture was vortexed for 2 seconds and then incubated at room temperature until gelation occurred, thereby providing a hydrogel bioink composition.

Hybrid hydrogels were prepared by mixing stock solutions of the respective peptides at the desired volume/volume ratios (e.g., 3:1, 1:1) following the procedure described above, to thereby provide the respective hybrid hydrogel bioink composition.

Gelatin-based composite (optionally hybrid) hydrogels (also referred to herein as gelatin-supplemented (optionally hybrid) hydrogels or gelatin-containing (optionally hybrid) hydrogel or composite (optionally hybrid) hydrogels) were prepared by dissolving gelatin in Dulbecco's modified Eagle's medium, for 2 hours at 37° C., at a concentration of 50 g·L¹ (50 grams/liter), to thereby provide a gelatin formulation, and thereafter adding a respective peptide stock solution (50 μL) to 950 μL of the gelatin solution, immediately followed by vortex mixing. The formed formulations thus included gelatin at a final concentration of 47.5 g·L⁻¹ and a respective peptide or mixture of peptides at a final concentration of 5 g L⁻¹, and provide respective composite (optionally hybrid) hydrogel bioink compositions.

Transmission electron microscopy (TEM): Samples (10 μl) were placed on copper grids (400 mesh) covered with a carbon-stabilized Formvar film (EMS, BNFFA1000-Cu). After 2 minutes, excess gel was removed, and the grids were negatively stained with 10 μl of UranylLess staining solution for 1 minute. Finally, excess fluid was removed, and the samples were viewed in a JEM 1400plus electron microscope operating at 80 kV.

Scanning electron microscopy (SEM): Samples were placed on a glass coverslip and dried under vacuum. The samples were then coated with Au and viewed using a scanning electron microscope (JEOL, Tokyo, Japan).

Rheological measurements: Rheological analysis was performed using an AR-G2 controlled rheometer (TA Instruments, New Castle, DA, USA). Oscillatory strain (0.01-100%) and frequency sweep (0.01-100 Hz) were conducted on 100 μL samples of pre gelated disks, 8 mm in diameter. The measurement was conducted with an 8 mm geometry (resulting in a gap size of 1 mm) at room temperature, in order to define the viscoelastic region in which the oscillatory test was performed.

Time-sweep oscillatory analyses used a constant a frequency of 5 Hz and a strain of 0.5%. Freshly prepared samples (220 μL) were placed under a 20 mm parallel plate geometry (resulting in a gap of 0.6 mm) at room temperature to determine the G′ (storage modulus) and G″, (loss modulus) for each sample.

Thixotropic analysis was conducted to examine the recovery behavior of the bioink compositions (hydrogels, hybrid hydrogels, composite hydrogel and/or composite hybrid hydrogels). Prior to the measurements, the bioink compostions were placed in the rheometer for 6 hours to allow full gelation. Aₙ initial time sweep was performed at 0.5% strain and 5 Hz for 30 minutes followed by 100% strain at 5 Hz to break the respective (optionally composite and/or hybrid) hydrogels. After each breaking cycle, 1 hour of recovery was monitored at 0.5% strain and 5 Hz. This was repeated for a total of 4 cycles. Temperature dependence studies were performed using temperature sweep (oscillation) by increasing the temperature from 15° C. to 45° C. at a heating rate of 1° C./minute. In order to evaluate the effect of temperature variation after the printing, bioink compositions were used to print a disk shape 1 mm in diameter and 1 mm in height and analyzed as described.

Absorbance kinetics of the hydrogel formation: Samples of 100 μL of Fmoc-FF, PegA, PepB formulations and the co-assembled hydrogels (bioink compositions) were placed into a 96-well plate. Absorbance at 400 nm was measured every 30 seconds using a TECAN Infinite M2000PRO plate reader for a total of 4 hours.

3D-printing: The 3D-printing was performed using a commercially available 3D bioprinter (Cellink BioX 3D BioPrinter, Cellink AB, Gothenburg, Sweden). Constructs (objects) were printed through a 22 G or 27G nozzle. Pressure and speed were optimized for each hydrogel composition, with the printbed at a constant temperature of 15° C., and a temperature-controlled printhead utilized to keep and print the hydrogels at 22° C.

Images of the printed constructs (objects) were captured using a zoom stereomicroscope Nikon SMZ800N equipped with a DS-Fi2 camera (Nikon, Japan).

To evaluate the printability of the hydrogel compositions, the filament spreading ratio was calculated by dividing the width of the printed filament by the nozzle diameter (410 μm). A second criterion for measuring the printability of the hydrogel compositions was applied as described, for example, in Ouyang et al. [Biofabrication, 2016, 8 035020], which introduces the printability parameter (Pr).

Briefly, the calculation is based on Equation (i), of circularity of enclosed areas:

$$C = 4 π AL - 2 \qquad Equation ( i )$$

where L is the perimeter and A is the area.

Circles are considered to have a circularity of C=1, while the circularity of a square is equal to α/4. Indeed, to define the square shape of a 3D-printed ink, using eEquation (ii):

$$Pr = π C - 1 / 4 = L 2 A - 2 / 16 \qquad Equation ( ii )$$

According to this equation, when the Pr is equal to 1, the construct prints as a complete square. To evaluate the Pr of the tested hydrogels, optical images of printed constructs (objects) were acquired and analyzed using NIS-Elements Viewer program.

Injectability test: A composite hydrogel Fmoc-FF/Gel and a hydrogel Fmoc-FF were prepared as described herein, with the addition of a blue food colorant, and were immediately loaded into a 1 mL syringe. Two hours thereafter, upon gelation, the samples were injected through a 27 G needle into a 20 mL glass vial containing double distilled water (DDW).

Cell viability: Fibroblast NIH3T3 cells) and OP9M2 murine stem cells were cultured in Dulbecco's modified Eagle's medium (DMEM) supplemented with 10% fetal calf serum, 100 U·mL⁻¹ penicillin, 100 U·mL⁻¹ streptomycin, and 2 mmol·L⁻¹ L-glutamine was utilized to grow the NIH3T3 fibroblasts, while Minimum Essential Medium-α (α-MEM) supplemented with 20% fetal calf serum and penicillin and 100 U·mL⁻¹ was utilized to grow the OP9M2 murine stem cells (all from Biological Industries, Israel). Cells were maintained in an incubator at 37° C. in a humidified atmosphere containing 5% CO₂.

Cell viability on 3D-printed scaffolds was performed by printing the hydrogel compositions (including hybrid hydrogels and composite, optionally hybrid, hydrogels) in 6-well plates. After about 24 hours post-printing, the printed objects were sterilized by UV, and then washed with DMEM or α-MEM for 2 hours on an orbital shaker at room temperature. Afterwards, two additional 2 hours washes were performed in the incubator, followed by a final overnight wash in the incubator (37° C., 5% CO₂). The cells were then seeded on a 6-well plate, on the 3D printed object (300,000 cells for NIH3T3 and 150,000 cells for the OP9M2 were seeded in 3 mL of their respective medium), while each experiment had six repeats for NIH3T3 cells and four repeats for OP9M2 cells.

AlamarBlue™ assay: At 24 hours- and 48 hours—post seeding, the cells were stained by replacing the cell medium with alamarBlue™ diluted 1:9 in the respective medium and incubating for 3 hours (37° C., 5% CO₂). Then, fluorescence was measured using a Tecan Spark plate reader at λex=540 nm and λem=590 nm. The results are presented as the percentage of viable cells relative to the control group of normal cell culture in a 6 well-plate.

A LIVE/DEAD™ staining solution containing 6.6 μg·mL⁻¹ of fluorescein diacetate and 5 μg·mL⁻¹ of propidium iodide was used to qualitatively evaluate the viability of cells on the 3D-printed constructs. Images were obtained using a confocal microscope ZEISS LSM 900 (ZEISS, Germany).

Statistical analysis: Statistical analysis was performed by using the GraphPad Prism Software (GraphPad Software, Inc., San Diego, CA, USA). Data for each assay were analyzed with one-way analysis of variance (ANOVA). Statistical significance was set at p<0.05 (*), and p<0.0001 (****).

### Example 1

### Design and Characterization of Peptides and Bioinks

Low molecular weight hydrogel-forming materials (gelators) have demonstrated excellent capability to self-assemble into supramolecular structures and form mechanically stable hydrogels [L. Adler-Abramovich and E. Gazit, Chem. Soc. Rev., 2014, 43, 6881-6893; D. J. Adams, Macromol. Biosci., 2011, 11, 160-173; Tomasini and Castellucci, Chem. Soc. Rev., 2013, 42, 156-172; G. Fichman and E. Gazit, Acta Biomater., 2014, 10, 1671-1682]. These hydrogels are held together by non-covalent interactions such as hydrogen bonds, Van der Waals forces, electrostatic interactions, and 7L-7L stacking [G. Fichman and E. Gazit, 2014, supra; Schweitzer-Stenner and Alvarez, J. Phys. Chem. B, 2021, 125, 6760-6775]. Compared to polymer-based hydrogels, hydrogels based on low molecular weight hydrogel-forming materials (gelators) require a lower concentration [G. Fichman and E. Gazit, Acta Biomater., 2014, 10, 1671-1682; Hauser and Zhang, Chem. Soc. Rev., 2010, 39, 2780-2790], resulting in lower level of bioaccumulation in the body which facilitates the process of biodegradation [G. Fichman and E. Gazit, 2014, supra]. Moreover, their low cost and the ability to easily design and decorate the building blocks make them good candidates to serve as scaffolds in tissue engineering applications [Dias and Peng, Physiol. Behav., 2017, 176, 139-148; Rajbhandary et al., Langmuir, 2017, 33, 5803-5813; Abraham et al., Langmuir, 2019, 35, 14939-14948].

The Fmoc-FF dipeptide is a low molecular weight hydrogel-forming material (gelator) that can form hydrogels composed of a fibrillary nanometric network [Dudukovic and Zukoski, Langmuir, 2014, 30, 4493-4500; Kamada et al., Small, 2019, 16, 9, 1904190; Adler-Abramovich et al., ACS Nano, 2016, 10, 11]. Numerous studies have described the remarkable characteristics of the Fmoc-FF hydrogel, such as its anti-inflammatory properties and its high rigidity which enhances cell adhesion [Zhou et al., J. Tissue Eng., 2014, 5, 1-7; Aviv et al., ACS Appl. Mater. Interfaces, 2018, 10, 41883-41891; Ghosh et al., Nanomaterials, 2019, 9(4), 497].

In order to avoid post-manufacturing steps or procedures in 3D-bioprinting processes, new techniques and bioink compositions are needed. The use of peptides for 3D bioprinting was recently suggested as a potential solution [Raphael et al., 2017, supra; Rauf et al., 2021, supra].

Due to their simple synthesis, biocompatibility, low-cost and notable mechanical properties, the present inventors have considered Fmoc-FF peptide hydrogels and other aromatic peptides bearing an end-capping moiety as candidate components in bioink formulations or compositions. However, Fmoc-FF peptides-based compositions lack the necessary injectability properties, as shown below.

In order to increase the solubility and stability of the hydrogel in the composition, as well as enhance its physiochemical properties, composite or hybrid hydrogels were targeted, in particular those containing polymers and self-assembling peptides [Dias and Peng, 2017, supra; Ryan et al., 2011, supra]. Such composite or hybrid hydrogels have been previously studied as candidates for various applications in the fields of drug delivery, encapsulation of cells, tissue engineering and regenerative medicine. Conjugation of polyethylene glycol as a synthetic, biocompatible, hydrophilic polymer, to peptide-based hydrogel forming agents, has been described in the context pharmaceutical applications [Rudnick-Glick et al., J. Nanobiotechnology, 2015, 13, 1-8; Ryan et al., 2011, supra; Diaferia et al., 2018, supra; Diaferia et al., 2019, supra] The effect of conjugating an ethylene glycol (EG)-containing moiety to Fmoc-FF as an exemplary self-assembling, hydrogel-forming, aromatic peptide and its effect on the printability of the formed (optionally hybrid) hydrogel were therefore studied.

Two representative lengths of ethylene glycols were conjugated to Fmoc-FF: diethylene glycol (EG)2 and hexaethylene glycol (EG)6, resulting in two exemplary alkylene glycol-containing moiety conjugated to aromatic peptides (alkylene glycol peptide conjugates): Fmoc-FF-EG₂—COOH (PepA) and Fmoc-FF-EG₆-COOH (PepB), respectively, as illustrated in FIG. 1.

Hydrogels made of a single peptide and hybrid hydrogels made of two peptides were prepared using the solvent switch method by dissolving the monomers in DMSO and then diluting the stock solutions into water to a final concentration of 5 g-L.

As can be seen in FIGS. 2A-C, both ethylene glycol motifs hamper the ability of the Fmoc-FF peptides to self-assemble and to form a hydrogel. While the Fmoc-FF hydrogel was rigid and stable, PepA formed a weak hydrogel, and PepB did not form a hydrogel at all.

Transmission electron microscope (TEM) imaging was employed to evaluate the effect of ethylene glycol conjugation on the hydrogel morphology and fibrillary matrix formation, and the results are presented in FIG. 2D-F, revealing that the ethylene glycol motif adversely affected the dense fibrillary network of the Fmoc-FF hydrogel. This effect was inversely related to the length of the ethylene glycol motif; while the Fmoc-FF hydrogel was composed of relatively thick and long fibrils, 16 nm in diameter, the PepA fibrils were found to be narrower with a diameter of 9 nm, while PepB were exhibited only short fibrillar fragments.

To further examine the effect of the ethylene glycol motifs, co-assembly of Fmoc-FF with either PepA or PepB at stoichiometric ratios of 1:1 and 3:1, was practiced to form exemplary hybrid hydrogels. This was achieved by mixing the appropriate ratios of stock solutions prepared in DMSO, and then adding water to the mixture to a final peptide concentration of 5 g·L⁻¹.

As can be seen in FIG. 2A-C, in all cases, transparent and homogeneous 3D self-supporting hydrogel (FIG. 2A) and hybrid hydrogels (FIGS. 2B-C) were formed.

In contrast to the results observed by TEM analyses of PepA and PepB, when co-assembled, the hybrid hydrogels exhibited a fibrillary nanostructure similar to that of the pristine Fmoc-FF hydrogel.

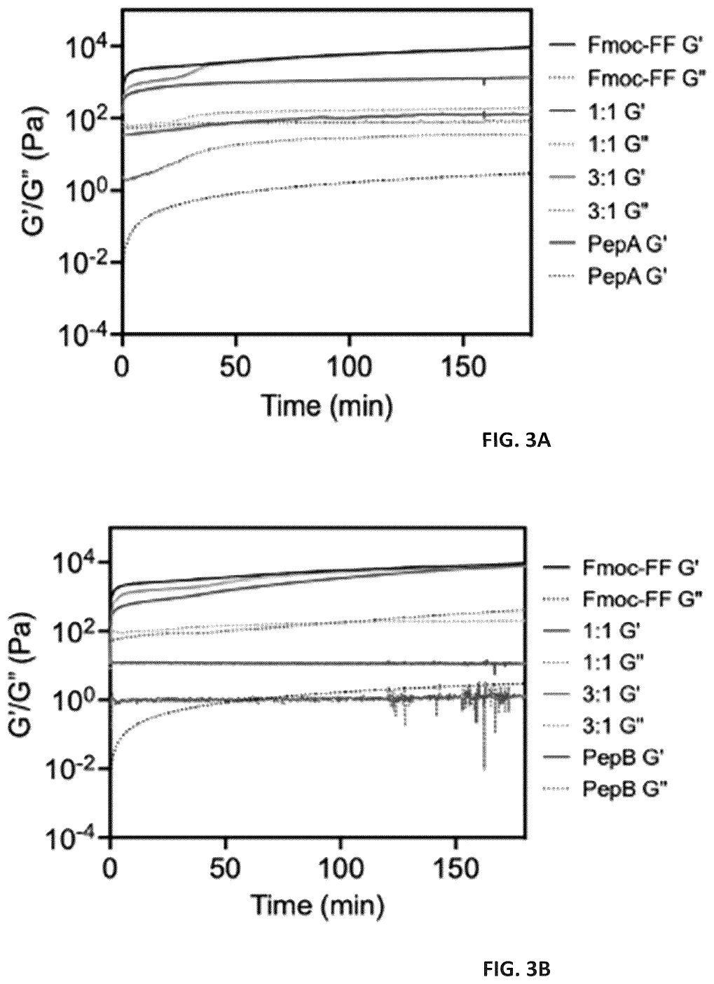

The in situ kinetics of the hydrogels formation and their mechanical properties were characterized by time sweep measurements over 4 hours at a fixed strain of 0.5% and frequency of 5 Hz, and the results are presented in FIGS. 3A-B.

The results indicated that while Fmoc-FF gelation occurred within 4 minutes, the ethylene glycol motif attenuated hydrogel formation, with the Fmoc-FF:PepA 1:1 and 3:1 hybrids reaching a plateau at about 50 minutes, as seen in FIG. 3A. The Fmoc-FF:PepB 1:1 and 3:1 hybrid hydrogels displayed a behavior similar to the pure Fmoc-FF, and reached a plateau of hydrogel formation after several minutes, as seen in FIG. 3B.

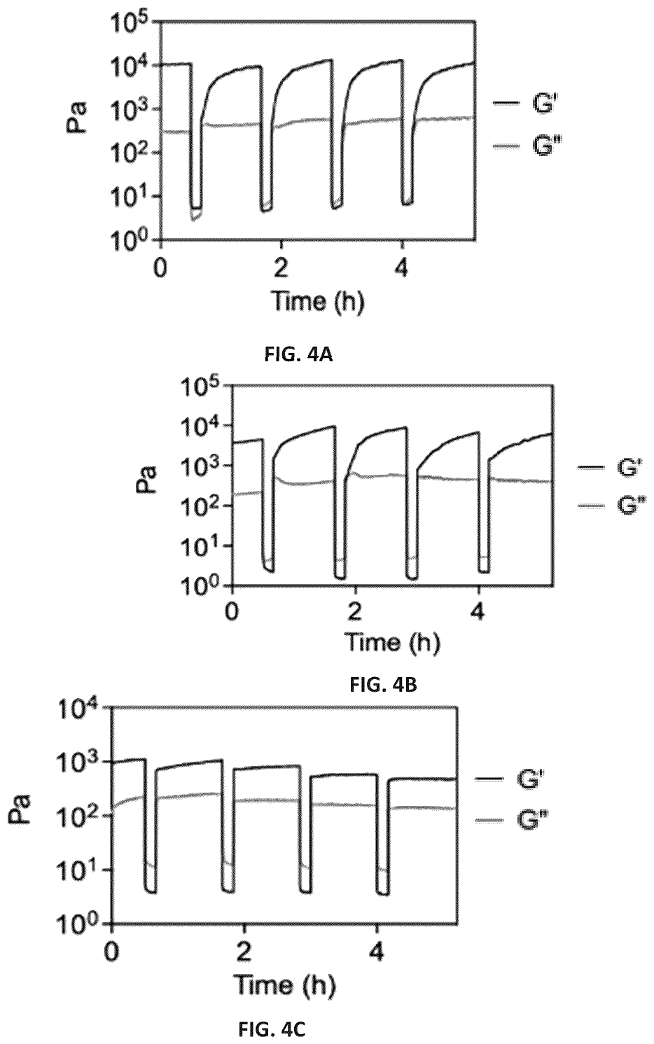

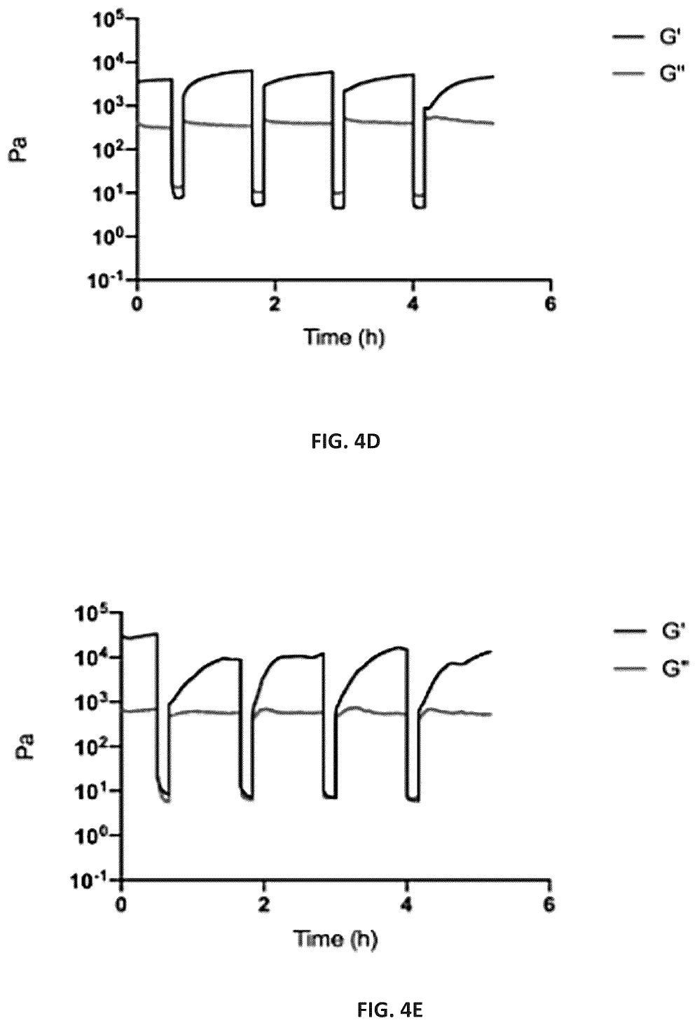

Rheological analysis was performed to compare the physical characteristics of the various hybrid hydrogels to the hydrogel formed by the self-assembly of pristine Fmoc-FF, and the results are presented in FIGS. 4A-C.

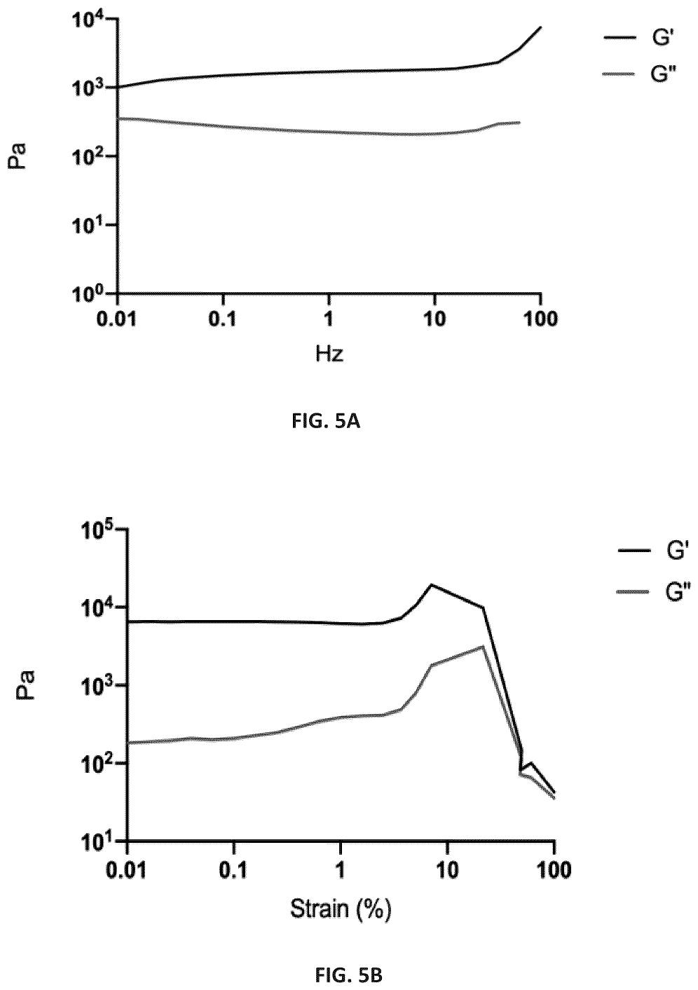

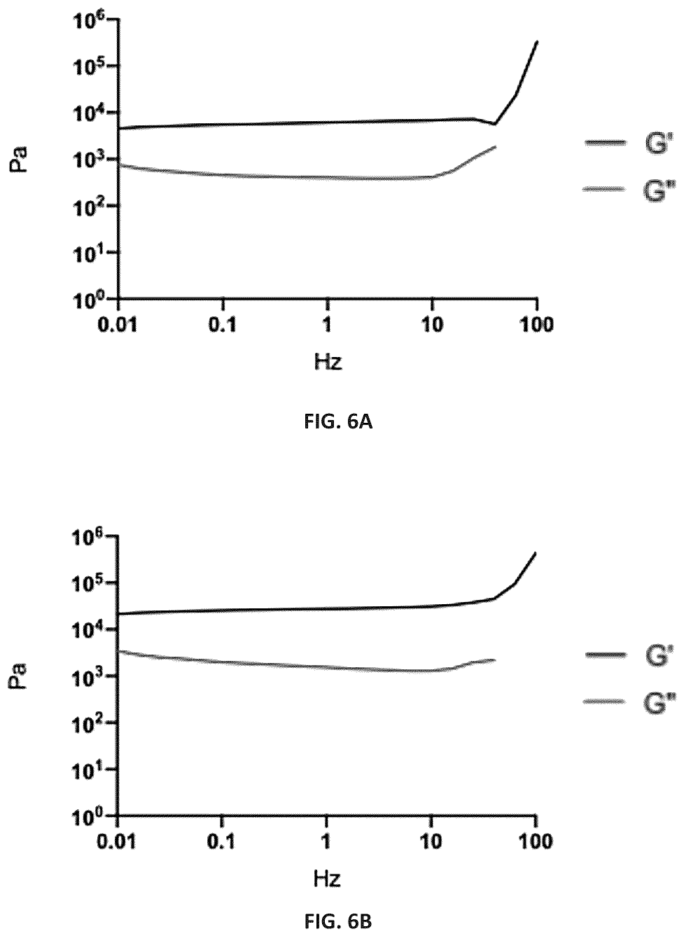

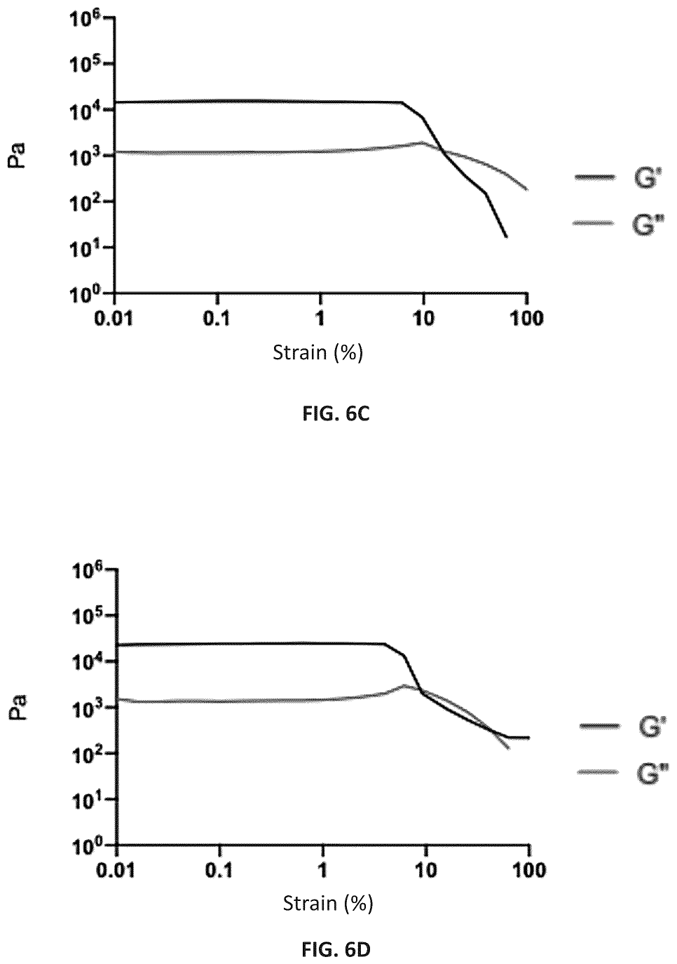

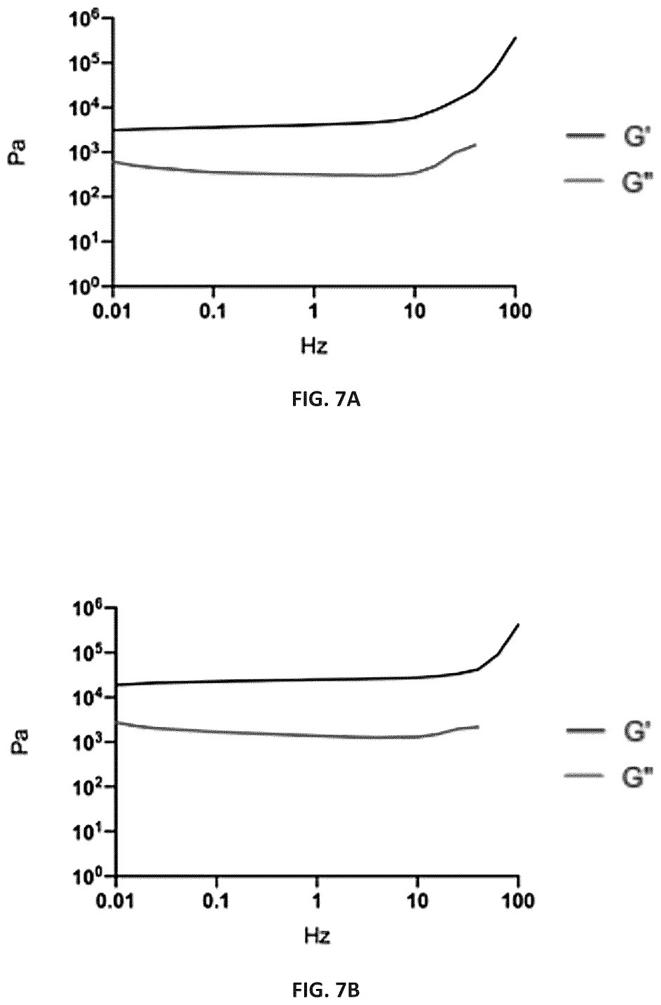

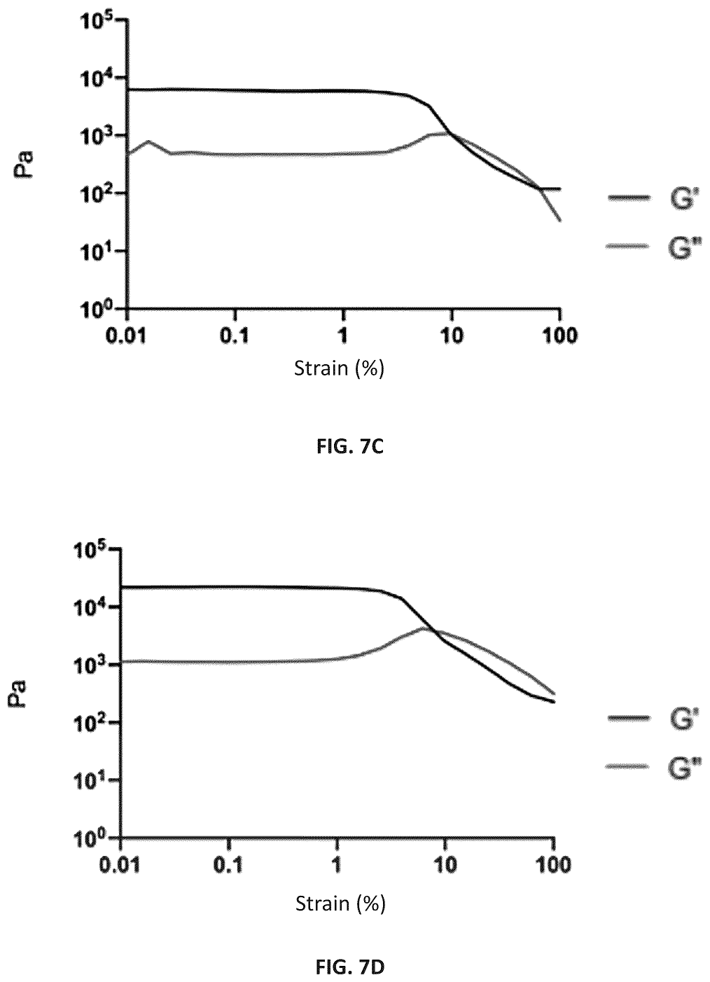

To determine the viscoelastic region, dynamic strain sweep (at 5 Hz) and frequency sweep (at 0.5% strain) oscillatory measurements were performed, and the results for Fmoc-FF, Fmoc-FF:PepA and Fmoc-FF:PepB are presented in FIGS. 5A-B, 6A-D and 7A-D, respectively.

The rheological profiles of the exemplary hydrogels are summarized in Table 1 herein:

TABLE 1

| Hydrogel | G′ [Pa] | G″ [Pa] | G′/G″ |
|---|---|---|---|
| Fmoc-FF | 9552 | 251 | 37 |
| PepA | 157 | 42 | 3 |
| PepB | 1 | 1 | 0 |
| 1:1 Fmoc-FF:PepA hybrid | 3788 | 286 | 13 |
| 1:1 Fmoc-FF:PepB hybrid | 10408 | 573 | 18 |
| 3:1 Fmoc-FF:PepA hybrid | 12301 | 240 | 51 |
| 3:1 Fmoc-FF:PepB hybrid | 9198 | 216 | 42 |

As can be seen in Table 1, comparing the storage modulus after 4 hours of gelation revealed that the storage modulus (G′) of each of the hybrid hydrogels, except the 1:1 Fmoc-FF:PepA hybrid hydrogel (3788 Pa), showed a similar value to Fmoc-FF (9552 Pa).

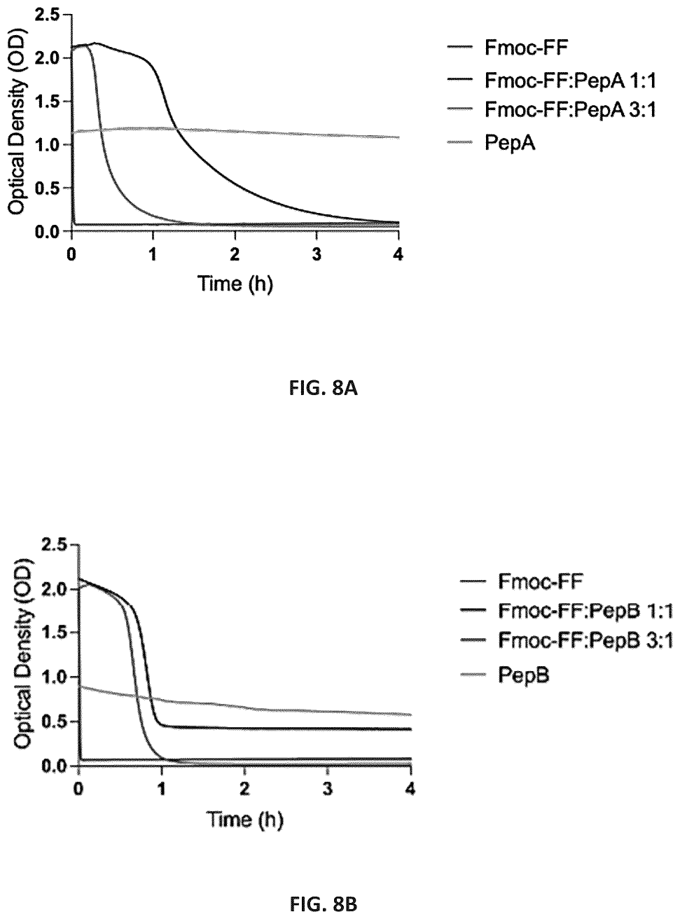

To further evaluate the impact of co-assembly on gelation kinetics, the absorbance spectra of the hybrid hydrogels compared to the Fmoc-FF hydrogel at 400 nm was monitored over time and the results are presented in FIGS. 8A-B.

While Fmoc-FF absorbance dropped to 0.1 OD within a few minutes, both the Fmoc-FF:PepA and Fmoc-FF:PepB hybrid hydrogels were much slower, requiring 90 minutes and 180 minutes, respectively, for the Fmoc-FF:PepA 3:1 and 1:1 hybrid hydrogels to form a clear hydrogel material with an OD value of 0.196. Similarly, the Fmoc-FF:PepB 3:1, and 1:1 hybrids hydrogels required 88 minutes and 60 minutes, respectively, with the latter only reaching a minimum OD value of 0.3.

These results support the findings that the conjugation of ethylene glycol to the Fmoc-FF peptide decreases the gelation kinetics and that the length of the ethylene glycol motif can influence the stability of the hydrogels.

Another fundamental property for 3D-printing applications of hydrogel-based bioink compositions is the capacity to recover after the stress of the 3D-printing process (thixotropy). In order to assess this ability, hydrogel formulations were placed under the rheometer and allowed to gelate for 6 hours in-situ in order to reach full gelation prior to thixotropic analysis. Aₙ initial time sweep analysis was then performed at 0.5% strain and 5 Hz for 30 minutes followed by 100% strain at 5 Hz in order to break the formed hydrogels. After each breaking cycle, an hour of recovery was monitored at 0.5% strain and 5 Hz.

As can be seen in FIGS. 4A-E, the exemplary hydrogel and hybrid hydrogels demonstrated thixotropic behavior even after four cycles, albeit with slight differences in the dynamics of the gelation recovery process. As can be seen, the 1:1 Fmoc-FF:PepB hydrogel (FIG. 4C) demonstrated an immediate recovery that was faster than the other tested samples.

As can be seen in FIGS. 9A-C, both Fmoc-FF:PepA and Fmoc-FF:PepB samples displayed low viscosity, similar to the Fmoc-FF hydrogel.

Overall, the obtained data demonstrate that it is possible to tune the mechanical properties of the peptide hydrogel by conjugating ethylene glycol moieties, as representative alkylene glycol-containing moiety, of different lengths, to the self-assembling hydrogel-forming peptides. When combining peptides conjugated to such moieties (i.e., PepA, PepB) with the pristine self-assembling peptides, the weight ratio between the self-assembling peptide (Fmoc-FF) and the conjugated peptide (i.e., PepA, PepB) affects the storage modulus in the formed hybrid hydrogels, such that increasing the ratio of PepA or PepB in the formulation resulted in a softer hybrid hydrogel (see, e.g., 1:1 Fmoc-FF:PepA and Fmoc-FF:PepB), while increasing the ratio of Fmoc-FF resulted in a stiffer hybrid hydrogel (see, e.g., 3:1 Fmoc-FF:PepA and Fmoc-FF:PepB).

### Example 2

### Design and Characterization of Composite Hydrogels

Recent studies have demonstrated that the incorporation of polymers into brittle hydrogels can provide the elasticity needed to overcome the shear stress applied during the 3D-printing process [Chimene et al., 2020, supra]. In the past decades, the biocompatible properties of gelatin prompted numerous studies on gelatin-based scaffolds for tissue engineering [Unagolla and Jayasuriya, Appl. Mater. Today, 2020, 18, 100479]. Furthermore, gelatin is a commonly used adjuvant in 3D-printing applications as it improves the viscosity and the elasticity of materials [Feng et al., 2019, supra; Godoi et al., 2016, supra].

Therefore, as schematically illustrated in FIG. 10, gelatin was incorporated into the peptide hydrogel or hybrid hydrogels as an exemplary matrix-forming biocompatible material in order to improve the elasticity of the hybrid Fmoc-FF:PepA and Fmoc-FF:PepB hydrogels. As demonstrated hereinafter, the introduction of the peptide hydrogel or hybrid hydrogels into the gelatin substantially increased the storage modulus of the gelatin hydrogel, rendering it suitable as a self-supporting, thermally-stable material for bioprinting.

The new formulation was produced by dissolving gelatin in DMEM at a concentration of 50 g·L⁻¹ and mixing the gelatin solution with a peptide solution as described herein, so as to achieve a composite hydrogel composition with gelatin at a final concentration of 47.5 g/L. The composite hydrogel compositions were printed 2 hours post-preparation with printhead and printbed temperatures of 22° C. and 15° C., respectively.

As can be seen in the photographs of the 3D-printed hydrogels shown in FIGS. 12A-C, the introduction of gelatin enhanced the elasticity properties of the hydrogels, facilitating 3D-printing.

Rheological studies were conducted to evaluate the influence of the gelatin on the novel bioink compositions. Aiming at evaluating the stiffness of the composite (optionally hybrid) hydrogels, time sweep oscillatory analyses were performed on freshly prepared samples and the results are presented in FIGS. 13A-C.

The analyses revealed that all the tested samples gelated in less than one hour, indicating that gelatin did not affect the gelation kinetics. In contrast, incorporation of gelatin did affect the stiffness, and was accompanied by a decrease in the storage modulus.

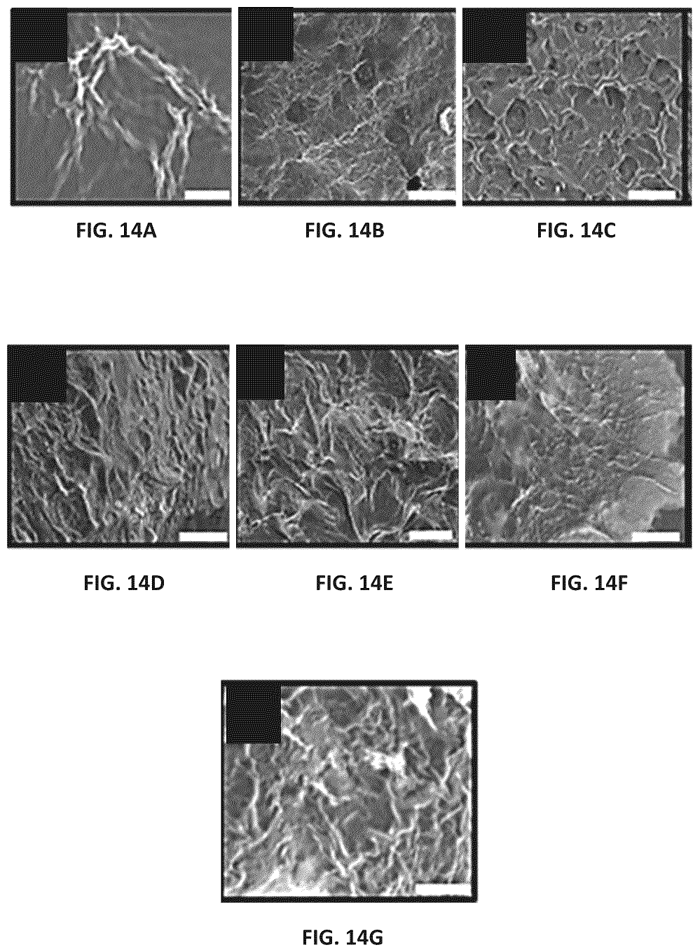

SEM imaging of the gelatin-supplemented composite hydrogels were performed. The results, presented in FIGS. 14A-H, revealed that unlike gelatin, which forms a porous amorphous matrix, the composite hydrogel and composite hybrid hydrogels exhibited a fibrillary matrix similar to that of the short peptide based hydrogels.

The rheological profiles of the exemplary composite hydrogels at 37° C. are summarized in Table 2 herein:

TABLE 2

| Hydrogel | G′ [Pa] | G″ [Pa] | G′/G″ |
|---|---|---|---|
| Fmoc-FF/Gel | 1416.5 | 241.102 | 5.875 |
| Fmoc-FF:PepA(1:1)/Gel | 1032.73 | 240.157 | 4.3 |
| Fmoc-FF:PepB | 1107.95 | 306.985 | 3.609 |
| (1:1)/Gel | | | |
| Fmoc-FF:PepA | 3573.46 | 695.53 | 5.137 |
| (3:1)/Gel | | | |
| Fmoc-FF:PepB | 2291.29 | 501.308 | 4.57 |
| (3:1)/Gel | | | |

The temperature effect on the storage modulus (G′) of the composite (optionally hybrid) hydrogels was evaluated both prior and post printing. For the non-printed samples, disk-shaped samples 8 mm in diameter and 1 mm in height with a volume of 100 μL were prepared in a silicon mold with the diameter of 8 mm and allowed to gelate overnight. The printed hydrogels were prepared by printing a full disk 10 mm in diameter and 1 mm in height and allowing it to re-gelate post-printing overnight. The experiment was performed using an 8 mm geometry with 0.5% strain and 5 Hz. The temperature was increased from 15° C. to 45° C. at a rate of 1° C./minute.

The obtained data is presented in FIGS. 15A-F, and show that while the storage modulus of pure gelatin dramatically dropped at 30° C. with the increase in temperature (FIG. 15A), the composite (optionally hybrid) hydrogels, including Fmoc-FF/Gel, Fmoc-FF:PepA (3:1 or 1:1)/Gel, or Fmoc-FF:PepB (3:1 or 1:1)/Gel, all exhibited notable thermostability (FIGS. 15B-F).

As can be seen in FIG. 15G, while the storage modulus of pure gelatin dropped dramatically at a temperature above 30° C., incorporation of gelatin in the Fmoc-FF, Fmoc-FF:PepA and Fmoc-FF:PepB hydrogels caused only a slight decrease in the storage modulus at higher temperatures, while maintaining a 3D self-supporting structure.

Similar results were observed upon incubation of the composite hydrogel and composite hybrid hydrogels at 37° C. for 20 minutes, as can be seen in the photographs presented in FIG. 16.

Comparing FIGS. 15A-C and FIGS. 15D-F, it can be seen that the printing process did not affect the physical properties of the composite hydrogel and composite hybrid hydrogels, which displayed a similar behavior to the composite hydrogel and composite hybrid hydrogels, respectively, before printing. These results suggest that the 3D-printing process did not affect the rheological properties, the storage modulus, or the thermostability of the composite hydrogel and composite hybrid hydrogels.

Overall, the addition of gelatin to the formulation enhanced the printability of both the hybrid hydrogels and the Fmoc-FF hydrogel.

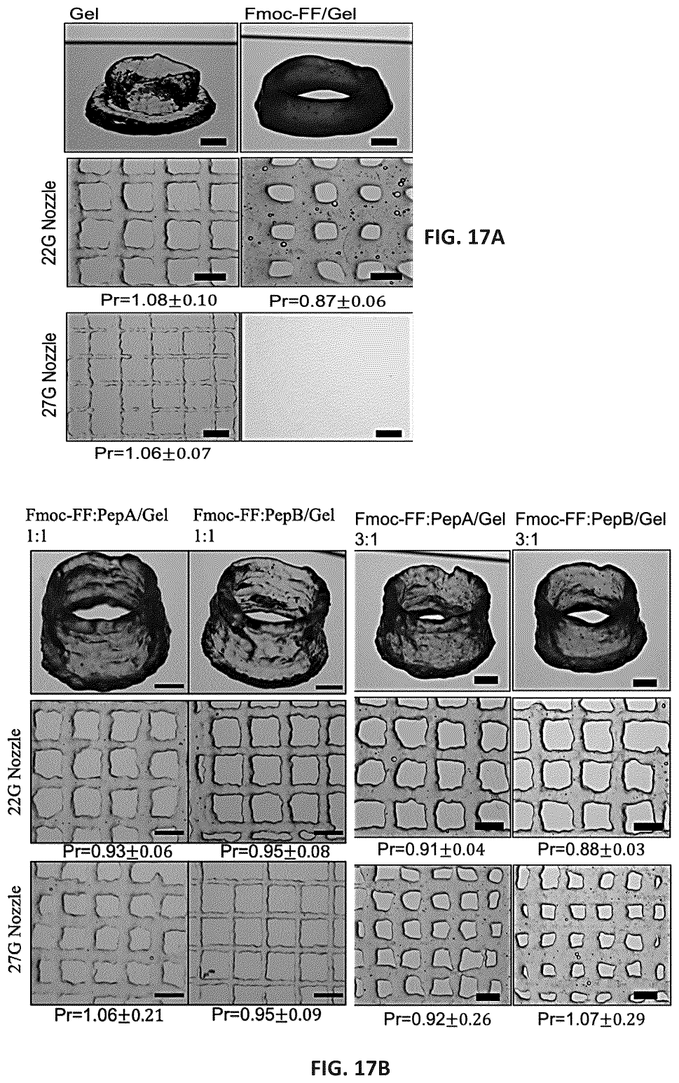

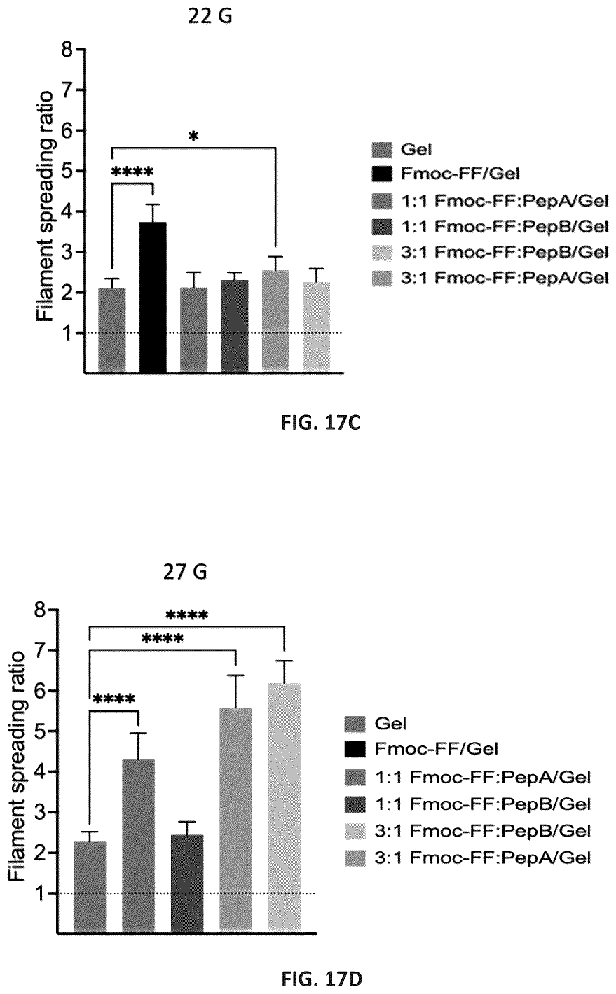

The printability of the composite hydrogel and composite hybrid hydrogels was further evaluated. The pressure and speed which enabled printing through a 22 and 27 G nozzle were adjusted according to the requirements of the different hydrogel compositions. 3D-printing of the Fmoc-FF/Gel was limited and required a number of adjustments during the printing process due to frequent obstruction of the nozzle. When printing an 11-layers cylindrical construct 5 mm in height, as can be seen in FIG. 17A, the Fmoc-FF/Gel composite hydrogel did not complete the 3D-printing process due to the obstruction of the 27 G nozzle.

However, as shown in FIG. 17B, the accuracy of the 3D-printing construct significantly improved in positive correlation with the concentration of the exemplary ethylene glycol-peptide conjugates PepA and PepB, while Gelatin alone was printable but did not show enough self-support to hold the shape.

These results demonstrate a synergistic effect of gelatin and the ethylene glycol-peptide conjugates. While the ethylene glycol peptide conjugates decrease the viscosity of hydrogels, as seen in FIGS. 9A-C, the matrix formed by the gelatin holds the 3D-printed filament shape.

The printability precision was then evaluated by filament-spreading ratio that ideally should be close to 1. These evaluations are presented in FIG. 17C.

As can be seen, measurements of the filament spreading ratio of the printed objects indicate that the exemplary composite hydrogels Fmoc-FF:PepA (1:1)/Gel and Fmoc-FF:PepB (1:1)/Gel provided the most precise 3D-printing outcome. While Fmoc-FF/Gel showed nozzle poor printability properties even when using the 22 G, exhibiting a filament spreading ratio of 3.7, the composite hybrid hydrogels demonstrated similar printability to the Gelatin itself with a filament spreading ratio values of 2.1, 2.6, 2.3 and 2.3 for 1:1 and 3:1 Fmoc-FF:PepA/Gel and 1:1 and 3:1 Fmoc-FF:PepB/Gel, respectively.

To further evaluate the printability properties of the composite hydrogels, a 27 G nozzle was also utilized. The results are also presented in FIGS. 17A-B and FIG. 17D.

The results show that while it was possible to 3D-print the exemplary composite hybrid hydrogels, the Fmoc-FF/Gel could not be extruded. The filament spreading ratio obtained from the 27 G nozzle showed less accurate 3D-printed outcome for most of the hydrogels, except for the 1:1 Fmoc-FF:PepB/Gel that did not show significant difference compared to Gelatin alone.

Evaluation of the printing precision showed that Fmoc-FF/Gel displayed a lower precision compared to the composite hybrid hydrogels. Therefore, the presence of ethylene glycol-peptide conjugates contributed to the precision of the 3D-printing of the composite hybrid hydrogels, and to the fabrication of constructs (objects).

Herein throughout, whenever a hybrid hydrogel or a composite hybrid hydrogel is referred to, the indicated ratio, e.g., 1:1 or 3:1 refers to the ratio of the two indicated peptides. Thus, for example, 1:1 Fmoc-FF:PepB is also referred to interchangeably as Fmoc-FF:PepB (1:1), 3:1 Fmoc-FF:PepB is also referred to interchangeably as Fmoc-FF:PepB (3:1), 1:1 Fmoc-FF:PepA is also referred to interchangeably as Fmoc-FF:PepA (1:1), and 3:1 Fmoc-FF:PepA is also referred to interchangeably as Fmoc-FF:PepA (3:1).

### Example 3

### Biocompatibility

The biocompatibility of the composite hydrogels was evaluated.

For this purpose, exemplary composite hydrogels of Fmoc-FF:PepA (3:1)/Gel and Fmoc-FF:PepB (3:1)/Gel were 3D-printed in a 6-well plate in a disk geometry 15 mm in diameter and 1 mm in height, with an infilled grid-pattern of 40%. The formulations were allowed to gelate for 2 hours prior to 3D-printing. 3D-printing was performed as described and the formed object was allows to sit overnight at room temperature, and then washed with DMEM or α-MEM before the cells were seeded.

In order to examine the biocompatibility of the printed scaffold, two cell lines were utilized, the murine fibroblast NIH3T3 and the mesenchymal stem cell OP9M2. Cell viability was evaluated after 24 hours and 48 hours using the alamarBlue™ assay.

As can be seen in FIG. 18A, NIH3T3 fibroblast seeded on both exemplary hydrogels demonstrated high viability, with 95% and 90% viability on Fmoc-FF:PepB (3:1)/Gel, and 78% and 100% viability on Fmoc-FF:PepA (3:1)/Gel after 24 hours and 48 hours, respectively. However, the composite hybrid hydrogels degraded over time. After 48 hours, the Fmoc-FF:PepB (3:1)/Gel was almost completely dissolved, while the Fmoc-FF:PepA (3:1)/Gel was still present in the plate but had lost its initial shape.

Similar results were obtained for the MSC OP9M2, as can be seen in FIG. 18B, with 92% and 110% viability on Fmoc-FF:PepB (3:1)/Gel, and 90% and 92% viability on 3:1 Fmoc-FF:PepA (3:1)/Gel after 24 hours and 48 hours, respectively.

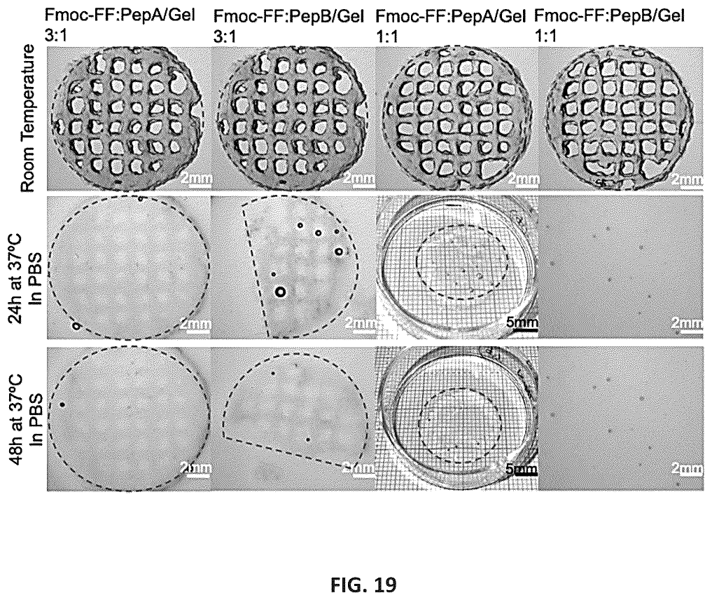

The stability of the 3D-printed scaffolds, was studied, following incubation at 37° C. in PBS. The results are presented in FIG. 19.

Immediately after printing, the scaffolds have a pink color, due to the presence of phenol red in the medium. After incubation in PBS the pink color decreased due to its diffusion out of the scaffold. While the Fmoc-FF:PepA (3:1)/Gel composite hybrid hydrogels showed good durability and maintained its 3D structure, the Fmoc-FF: PepB (3:1)/Gel demonstrated partial degradation. Moreover, the 1:1 composite hybrid hydrogels showed poor stability after incubation with PBS. The Fmoc-FF:PepA (1:1)/Gel composite hybrid hydrogel fully dissolved after incubation, while the Fmoc-FF:PepA (1:1)/Gel composite hybrid hydrogel lost the original shape and size.

The Fmoc-FF:PepA (3:1)/Gel and Fmoc-FF:PepB (3:1)/Gel composite hydrogels were further examined for their biocompatibility.

Further investigations included confocal microscopy to evaluate the presence and morphology of the NIH3T3 fibroblasts and the OP9M2 on the hydrogel constructs, and a LIVE/DEAD™ assay to identify live cells using fluorescein diacetate (green) and dead cells using propidium iodine (red).

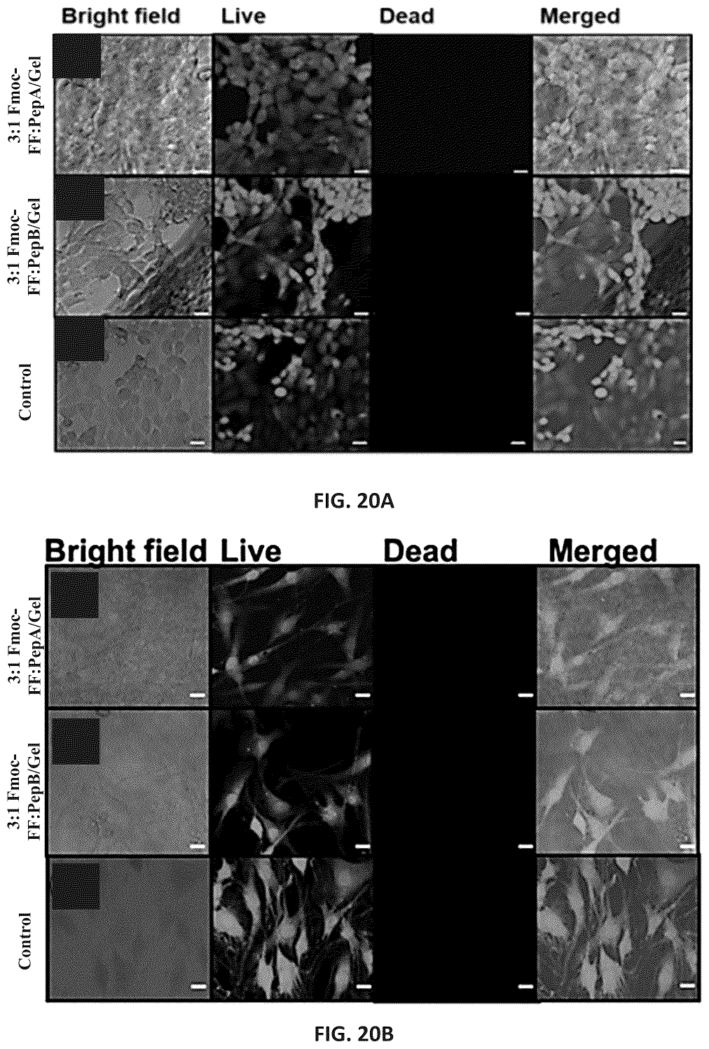

The results, presented in FIG. 20A-B, show that both NIH3T3 and MSC OP9M2 cells demonstrated high viability and were dispersed homogeneously on the exemplary composite hybrid hydrogels. These results demonstrate that the exemplary composite hybrid hydrogels are highly biocompatible.

Overall, the exemplary composite hybrid hydrogels show excellent mechanical and biocompatible properties that make them suitable for use as bioinks. Although the introduction of gelatin compromised the stiffness of the hydrogels and reduced its storage modulus, all the examined composite hydrogels were thermostable.

These composite hydrogels represent a new approach in the formulation of 3D-printing bioink that avoids the need for post printing processing, such as UV cross-linking, and therefore bear great potential for use fields such as tissue engineering.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A bioink composition comprising a matrix-forming biocompatible material and a self-assembled hydrogel formed of a plurality of peptides, wherein at least a portion of said plurality of peptides comprises aromatic peptides of 2 to 6 amino acid residues featuring an aromatic end-capping moiety.

2. The bioink composition of claim 1, wherein said plurality of peptides comprises at least one first portion that comprises said aromatic peptides of 2 to 6 amino acid residues featuring an aromatic end-capping moiety and at least a second portion that comprises aromatic peptides of 2 to 6 amino acid residues featuring an aromatic end-capping moiety and having an alkylene glycol-containing moiety conjugated thereto.

3. The bioink composition of claim 1, wherein at least a portion, or all, of said aromatic peptides in said first and/or second portion, if present, are aromatic dipeptides.

4. The bioink composition of claim 1, wherein at least a portion, or all, of said aromatic peptides in said first and/or second portion, if present, are aromatic homodipeptides.

5. The bioink composition of claim 4, wherein said aromatic homodipeptide is Phe-Phe.

6. The bioink composition of claim 4, wherein said aromatic homodipeptide is Fmoc-Phe-Phe.

7. The bioink composition of claim 3, wherein said alkylene glycol-containing moiety is an oligo(alkylene glycol) moiety of from 2 to 20, or from 2 to 10, or from 2 to 8, or from 2 to 6, alkylene glycol moieties.

8. The bioink composition of claim 1, wherein a weight ratio of said first and second portions ranges from 5:1 to 1:5, or from 3:1 to 1:3, or from 3:1 to 1:1.

9. The bioink composition of claim 1, wherein said matrix-forming biocompatible material is selected from a synthetic polymer, a naturally-occurring polymer, a protein and a carbohydrate.

10. The bioink composition of claim 1, wherein said matrix-forming biocompatible material is or comprises gelatin.

11. The bioink composition of claim 1, wherein a weight ratio of said matrix-forming agent and said self-assembled hydrogel ranges from 1:1 to 100:1.

12. The bioink composition of claim 1, further comprising a biological component other than said matrix-forming biocompatible material and/or said aromatic peptides.

13. A method of additive manufacturing a three-dimensional biocompatible object, the method comprising dispensing at least one bioink composition to sequentially form a plurality of layers in a configured pattern corresponding to a shape of the object,
wherein for at least a portion of said layers, said dispensing is of the bioink composition of claim 1,
thereby manufacturing the three-dimensional object.

14. A three-dimensional object prepared by the method of claim 13.

15. A three-dimensional object comprising a composition which comprises a matrix-forming biocompatible material and a self-assembled hydrogel formed of a plurality of peptides, said plurality of peptides comprising at least a first portion that comprises aromatic peptides of 2 to 6 amino acid residues featuring an aromatic end-capping moiety and at least a second portion that comprises aromatic peptides of 2 to 6 amino acid residues featuring an aromatic end-capping moiety and having an alkylene glycol-containing moiety conjugated thereto, the composition further comprising a biological component associated with said composition.

16. The three-dimensional object of claim 15, wherein said biological component comprises cells.

17. A formulation comprising a plurality of peptides which comprises at least a first portion comprising aromatic peptides of 2 to 6 amino acid residues featuring an aromatic end-capping moiety and at least a second portion comprising aromatic peptides of 2 to 6 amino acid residues featuring an aromatic end-capping moiety and having an alkylene glycol-containing moiety conjugated thereto, said plurality of peptides are capable of self-assembling to thereby form a hydrogel.

18. The formulation of claim 17, wherein a weight ratio of said first and second portions ranges from 5:1 to 1:5, or from 3:1 to 1:3, or from 3:1 to 1:1.

19. A method of preparing an ink composition that comprises a biocompatible matrix-forming material, the method comprising contacting the formulation of claim 17 and the biocompatible matrix-forming material with an aqueous solution.
